(19) United States
(12) Patent Application Publication
(10) Pub. No.: US 2023/0172959 A1
(43) Pub. Date: Jun. 8, 2023

(54) USE OF NMN FOR THE PREVENTION AND/OR TREATMENT OF A BACK PAIN AND CORRESPONDING COMPOSITIONS

(71) Applicant: NUVAMID SA, EPALINGES (CH)

(72) Inventors: Guillaume BERMOND, MARSEILLE (FR); Laurent GARCON, SAUSSET LES PINS (FR)

(73) Assignee: NUVAMID SA, EPALINGES (CH)

(21) Appl. No.: 17/910,581

(22) PCT Filed: Mar. 12, 2021
(86) PCT No.: PCT/EP2021/056318

§ 371 Date: Sep. 9, 2022

(30) Foreign Application Priority Data
Mar. 12, 2020 (FR) 2002475

(51) Int. Cl. A61K 31/706; A61K 9/00; A61K 31/7084; A61P 29/00; A61K 9/107; A61K 47/44; A61K 47/10; A61K 47/14; A61K 47/22

(52) U.S. Cl. CPC A61K 31/706; A61K 9/0014; A61K 31/7084; A61P 29/00; A61K 9/107; A61K 47/44; A61K 47/10; A61K 47/14; A61K 47/22

(57) ABSTRACT

Nicotinamide mononucleotide (NMN), a pharmaceutically acceptable derivative thereof, or a pharmaceutically acceptable salt thereof, are described for use thereof in the prevention and/or treatment of a back pain such as a lumbalgia (low back pain), a dorsalgia (severe back pain), or a cervicalgia (neck pain), preferably a chronic lumbalgia; as well as compositions that include the same.

### FIELD OF THE INVENTION

The present invention relates to the use of nicotinamide mononucleotide (NMN), a pharmaceutically acceptable derivative thereof, or a pharmaceutically acceptable salt thereof, as well as compositions that comprise the same, for the prevention and/or treatment of back pain, in particular lumbalgia (or low back pain) and chronic lumbalgia.

### TECHNICAL BACKGROUND

The vertebral column is made up of 24 vertebrae which include 7 cervical, 12 dorsal or thoracic, and 5 lumbar vertebrae, these being in addition to the sacrum and the coccyx which are the “fused” vertebrae. The cervical, dorsal and lumbar vertebrae are separated by intervertebral discs and are said to be “mobile”. The vertebral column is also connected to a set of ligaments and muscles.

Back pains, and more specifically pain in the vertebral column (spine), is an increasingly common ailment and may result from different factors such as having poor posture, carrying excessive loads, or as a consequence of an underlying pathology. Examples of pathologies that may be cited include: wear and tear on joints, pinching of the nerve roots, osteoporosis, crushed or herniated intervertebral discs, degeneration of the intervertebral discs, spinal deformity, tumours, trauma such as fracture following an accident or osteoporotic fracture, muscle pain and other causal conditions.

The most common types of pain in the vertebral column include pains felt in the cervical vertebrae (cervical spine) referred to as cervicalgia (neck pain) and pains felt in the lumbar vertebrae (lumbar spine) referred to as lumbalgia. Pain occurring in the thoracic vertebrae (thoracic spine) is less common and is referred to as dorsalgia (severe back pain).

The cervical vertebrae are the most vulnerable because they support and move the head. Cervicalgia neck pains are often caused by: osteoarthritis, that is to say, wear and tear on the vertebrae and the cartilage thereof; poor posture; pinching of the nerves between the vertebrae; a herniated disc; a trauma; or narrowing of the spinal canal.

Lumbalgia or low back pain is pain that occurs in the lumbar vertebrae. Lumbalgia is most often not serious. A sedentary lifestyle, carrying heavy loads in the course of professional activity, poor posture or poor movement are examples of causal factors that can induce lumbalgia. Indeed, the lumbar vertebrae are constantly subjected to stress (solicitation) and support a significant part of the body weight, thereby making the lumbar spine a particularly fragile region of the body.

There is a distinction made between acute and chronic lumbalgia (low back pain). Acute lumbalgia, commonly referred to as lumbago, or crick in the back or back strain, is a pain that could persist for up to 4 weeks and may disappear on its own. Chronic lumbalgia is a constant pain that persists for three months and longer.

Lumbalgia, whether acute or chronic, is merely a symptom of which the causes may be highly varied. Moreover these causes are difficult to determine. In 90% of cases, low back pain is benign and does not result from a major injury. Low back pain may be linked to damage or injury (lesions) to a muscle, a tendon or a ligament, for example resulting from exertion of an effort, an unusual twist, or the accumulation of micro-lesions caused by repetitive movements. Low back pain may also be caused by disc degeneration. With aging, the intervertebral discs lose their elasticity. While this degeneration is not always associated with pain, it may be involved in certain low back pains.

Lumbalgia or low back pain may also be caused by a herniated disc. It occurs when part of the gel-like substance in the intervertebral disc protrudes outward and compresses the nerve roots.

Low back pain may also be caused by a gynecological problem. Many women experience back pain on a periodic or constant basis, as a consequence of painful periods, endometriosis, etc. Thus although the source of the pain is not situated in the lumbar region, the pain however, still radiates to the lower back.

Low back pain can also be due to the sliding of one vertebra over another vertebra, referred to as spondylolisthesis. This situation may occur due to congenital weakness in the vertebral structures or as a result of a trauma.

Lower back pain may also be caused by arthritis, osteoarthritis or osteoporosis. If the osteoporosis of the vertebral column is significant, it can cause a vertebral fracture. Certain inflammatory arthritis, such as ankylosing spondylitis, may also cause pain and stiffness in the lower back. In rare cases, low back pain may be caused by an abdominal aortic aneurysm, a tumour, osteoporosis-related fracture, or an infection.

With regard to chronic lumbalgia or low back pain, the Haute Autorité de Santé/French National High Authority for Health distinguishes between three types: (i) non-degenerative lumbalgia previously referred to as specific lumbalgia or secondary lumbalgia, said to be symptomatic, linked to a trauma-induced, tumoral, infectious or inflammatory cause; (ii) degenerative lumbalgia, the origin of which may be a combination of one or more causes of the following types: discogenic or facet or mixed, ligamentary, muscular, linked to a regional or global disorder involving spinal (in)stability; and (iii) lumbalgia with no relationship to anatomic lesions. The term “discogenic lumbalgia” is understood to refer to a lumbalgia related to a pain in or damage to the intervertebral discs. The term “facet lumbalgia” is understood to refer to a lumbalgia caused by an injury or pain in the facet joints, that is to say, the moving joints in the vertebral column that connect the vertebrae to each other.

Regardless of the original cause of the low back pain, the pain is often linked to a contracture of the muscles around the lumbar vertebrae which is a reflex reaction to protect this anatomical area. This may then trigger a vicious cycle thereby contributing to the pain becoming chronic.

The treatment of such pains generally includes the administration of analgesics, non-steroidal anti-inflammatory drugs (NSAIDs), cortisone or cortisone derivatives, via topical, oral or injection routes.

However, the administration of these drugs causes damage to organs such as the stomach, the liver and the kidneys, among others. In addition, their effectiveness declines over time requiring increased doses. Furthermore, the chronic use of cortisone derivatives induces in particular bone fragility, neuropsychiatric effects, muscle wasting and lowering of immunity, thus leaving the patient vulnerable to infections.

There therefore exists a need to develop new compositions for the treatment and/or prevention of back pain, in the vertebral column, which serve to mitigate the drawbacks in the prior art.

### SUMMARY OF THE INVENTION

These objects are achieved by the invention as described below.

The present invention relates to nicotinamide mononucleotide (NMN), a pharmaceutically acceptable derivative thereof, or a pharmaceutically acceptable salt thereof, for use thereof via topical administration in the prevention and/or treatment of a back pain.

Advantageously, the pharmaceutically acceptable derivative of NMN may be dihydronicotinamide mononucleotide (NMN-H).

Advantageously, the pharmaceutically acceptable derivative of NMN may be alpha-NMN.

Advantageously, the pharmaceutically acceptable derivative of NMN may be selected from among: —a compound having the formula (I):

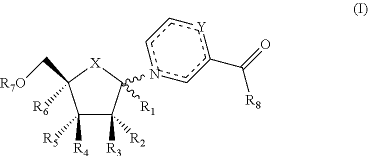

or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, P(S)R₉R₁₀ and

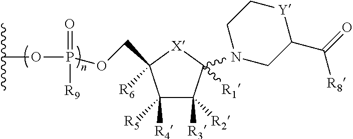

where n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂SO₂; wherein each of R₁₁ₐ is independently selected from H and a (C₁-C₆) alkyl, and R_{11b} is independently selected from OH, C₁-C₆ alkoxy, NH₂, NH(C₁-C₆alkyl) or N(C₁-C₆alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀ form, together with the phosphorus atoms to which they are attached, a 6-membered ring in which —R₉-R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃;
[symbol] represents a single or a double bond along Y; and
[symbol] represents the alpha or beta anomer depending on the position of R₁;
   or
   a compound having the formula (Ia):

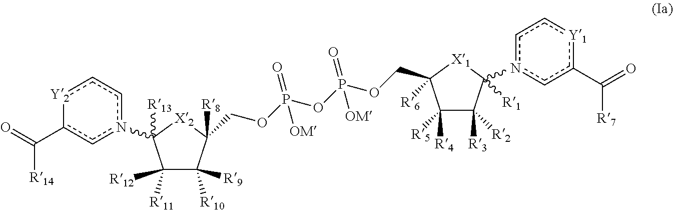

or one of the: stereoisomers, salts, hydrates, solvates, or crystals thereof, in which:
X′₁ and X′₂ are independently selected from among O, CH₂, S, Se, CHF, CF₂, and C═CH₂;
R′₁ and R′13 are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′₁ and Y′₂ are independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
and combinations thereof.

In a first preferred embodiment, the pharmaceutically acceptable derivative is the compound having the formula (I).

In one variant of the first embodiment, X represents an oxygen.

In one variant of the first embodiment, R₁ and R₆ each independently of one another represent a hydrogen.

In one variant of the first embodiment, R₂, R₃, R₄ and R₅ each independently of one another represent a hydrogen or an OH.

In one variant of the first embodiment, Y represents a CH.

In one variant of the first embodiment, Y represents a CH₂.

In one variant of the first embodiment, R₇ represents a hydrogen.

In one variant of the first embodiment, R₇ represents P(O)(OH)₂.

In one variant of the first embodiment,

X represents an oxygen; and/or
R₁ and R₆ each independently represent hydrogen; and/or
R₂, R₃, R₄ and R₅ each independently represent hydrogen, or R₂, R₃, R₄ and R₅ independently represent OH; and/or
Y represents a CH or a CH₂; and/or
R₇ represents P(O)R₉R₁₀, wherein R₉ and R₁₀ are independently selected from among OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred variant of the first embodiment, the compound of the invention is selected from among the compounds having the formula IB to IJ:

TABLE 1

| Compounds No | |
|---|---|
| (Anomers) | Structure |
| I-B (alpha) | 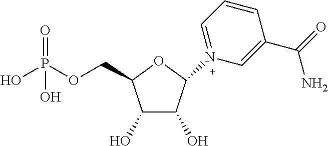 |
| I-C (beta) | 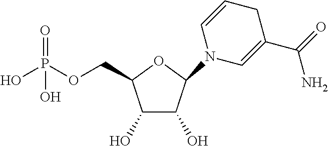 [SMILES: NC(=O)C1=CN([C@@H]2O[C@H](COP(=O)(O)O)[C@@H](O)[C@H]2O)C=CC1] |
| I-D (alpha) | 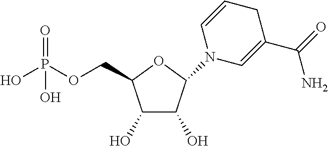 [SMILES: NC(=O)C1=CN([C@H]2O[C@H](COP(=O)(O)O)[C@@H](O)[C@H]2O)C=CC1] |
| I-E (beta) | 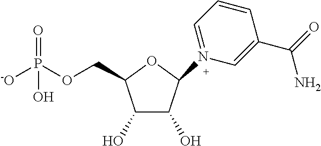 |
| I-F (alpha) | 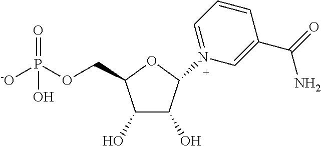 |
| I-G (beta) | 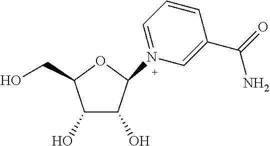 |
| I-H (alpha) | 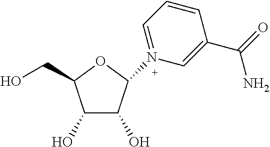 |
| I-I (beta) | 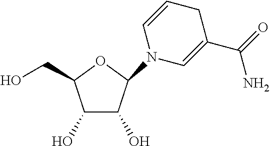 [SMILES: NC(=O)C1=CN([C@@H]2O[C@H](CO)[C@@H](O)[C@H]2O)C=CC1] |
| I-J (alpha) | 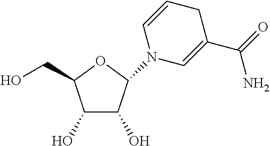 [SMILES: NC(=O)C1=CN([C@H]2O[C@H](CO)[C@@H](O)[C@H]2O)C=CC1] |

Advantageously, the pharmaceutically acceptable derivative of NMN may be alpha-NMN compounds I-F).

Advantageously, the pharmaceutically acceptable derivative of NMN may be dihydronicotinamide mononucleotide (NMN-H) (compounds I-C or I-D).

In a preferred second embodiment, the pharmaceutically acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula Ia-A to Ia-I:

TABLE 2

| Compounds | |
|---|---|
| (Anomers) | Structure |
| Ia-A (beta, beta) | 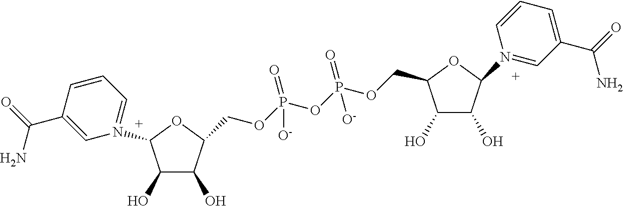 |
| Ia-B (beta, alpha) | 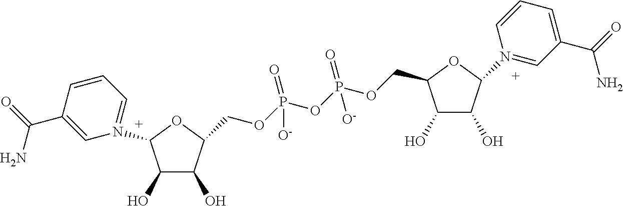 |
| Ia-C (alpha, alpha) | 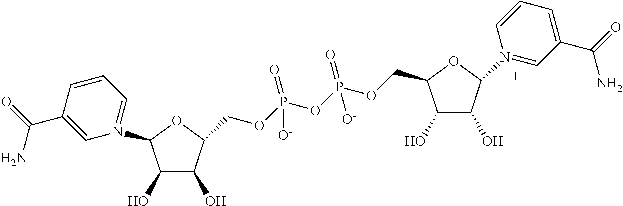 |
| Ia-D (beta, beta) | 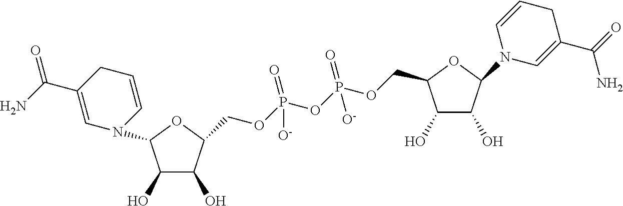 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 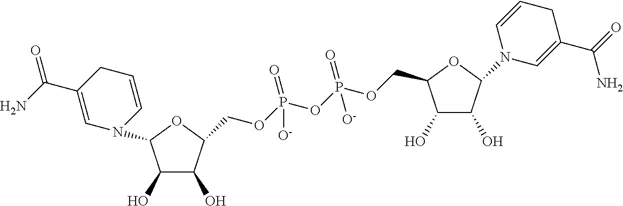 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 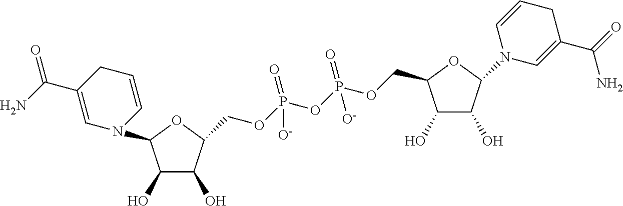 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 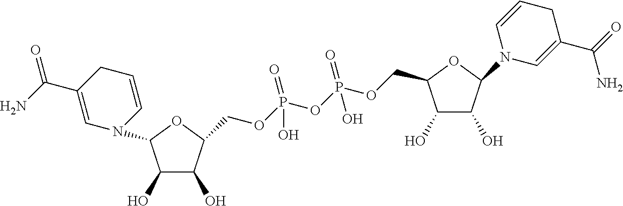 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 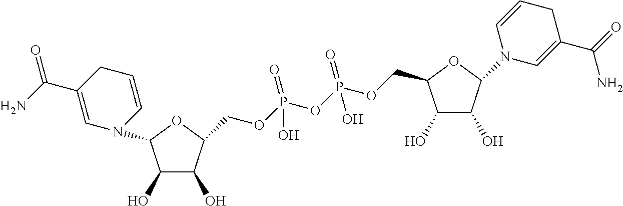 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 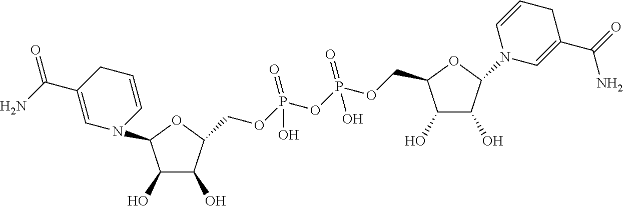 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

Advantageously, the back pain may be a cervicalgia (neck pain), a dorsalgia (severe back pain), or a lumbalgia (low back pain). Preferably, the back pain is a cervicalgia or a lumbalgia. On a more preferred basis, the back pain is a chronic lumbalgia.

Advantageously, the back pain may be due to one of the pathologies selected from among: injury to a muscle, injury to a ligament, injury to a tendon, degeneration of the intervertebral discs, a herniated disc, a pain that is gynecological in origin, spondylolisthesis, arthritis, osteoarthritis, osteoporosis of the vertebral column (or spine), osteoporosis-related fracture, an abdominal aortic aneurysm, a tumour, an infection, an inflammation, facet joint injuries, intervertebral disc injuries, regional or global spinal (in)stability-related [spinal statics] disorders, spinal deformity, muscular contraction in the vertebrae, or combinations thereof.

Advantageously, the back pain may be classified within one of the categories of the International Classification of Diseases ICD-10, preferably within the categories M40 to M43; M46 to M54 and G55.

Preferably the back pain is a lumbalgia or low back pain, and more preferably a chronic lumbalgia.

On a more preferred basis, the back pain or the lumbalgia are due to an inflammation, muscle contracture, muscle tear, ligament injury, tendon injury or combinations thereof.

Advantageously, the lumbalgia or low back pain may be classified under one of the categories M50 to M54 and G55.1, preferably under the categories M51, M54 and G55.1, of the International Classification of Diseases ICD-10.

Advantageously, the NMN, a pharmaceutically acceptable derivative thereof, or a pharmaceutically acceptable salt thereof, is intended to be administered between 1 and 10 times per day, preferably between 1 and 5 times per day, more preferably between 1 and 3 times a day.

In an even more preferred embodiment, the NMN, a pharmaceutically acceptable derivative thereof, or a pharmaceutically acceptable salt thereof, is intended to be administered twice a day.

Advantageously, the NMN, a pharmaceutically acceptable derivative thereof, or a pharmaceutically acceptable salt thereof, may be used in combination with at least one other therapeutic agent.

Advantageously, the at least one therapeutic agent may be an analgesic, a non-steroidal anti-inflammatory drug, cortisone, a cortisone derivative, a muscle relaxant, or combinations thereof.

Advantageously, the analgesic may be selected from among paracetamol, nefopam, ketanin, tetrahydrocannabinol, cannabinoids, aspirin, methyl salicylate, diflunisal, salicylamide, codeine, alfentanil, carfentanil, dihydrocodeine, codeinone, tramadol, morphine, morphinone, buprenorphine, fentanyl, acetyl fentanyl, remifentanil, sufentanil, heroin, hydromorphone, nalbuphine, oxycodone, hydroxycodone, oxymorphone, laudanum, methadone, pethidine, dextropropoxyphene, endorphin, tapentadol, thebaine, vicodin, and combinations thereof.

Advantageously, the non-steroidal anti-inflammatory drug may be selected from among ibuprofen, ketoprofen, naproxen, ketorolac, alminoprofen, aceclofenac, mefenamic acid, niflumic acid, tiaprofenic acid, celecoxib, rofecoxib, valdecoxib, parecoxib, dexketoprofen, diclofenac, etodolac, etoricoxib, fenoprofen, flurbiprofen, indomethacin, meloxicam, nabumetone, piroxicam, sulindac, tenoxicam, nimesulide, and combinations thereof.

Advantageously, the cortisone derivative may be selected from among betamethasone, ciprofloxacin, cortivazol, dexamethasone, fludrocortisone, methylprednisolone, prednisolone, triamcinolone, and combinations thereof.

Advantageously, the muscle relaxant may be selected from among centrally acting muscle relaxants, peripherally acting muscle relaxants, direct acting muscle relaxants, and combinations thereof.

The carbamic esters may be methocarbamol.

Advantageously, the peripherally acting muscle relaxants may be selected from among blockers (inhibitors) of acetylcholine release at the neuromuscular junction such as botulinum toxin type A and botulinum toxin type B, voltage gated sodium channel blockers such as conotoxins and huwentoxins, voltage-gated calcium channel blockers such as dihydropyridines, of muscle nicotinic acetylcholine receptor blockers such as curares or conotoxins.

Advantageously, the direct-acting muscle relaxant is a ryanodine receptor blocker such as dantrolene.

Advantageously, the muscle relaxant may also be selected from among baclofen, quinine, mephenesin, tizanidine, tetrazepam, thiocolchicoside, acetyl hexapeptide-8, μ-conotoxin CnIIIc (mu-conotoxin CnIIIc), dipeptide diaminobutyroyl benzylamide diacetate as well as locally used botulinum toxin, and combinations thereof.

Advantageously, nicotinamide mononucleotide (NMN), a pharmaceutically acceptable derivative thereof, or a pharmaceutically acceptable salt thereof, makes it possible to reduce the stiffness in the vertebral column.

Advantageously, nicotinamide mononucleotide (NMN), a pharmaceutically acceptable derivative thereof, or a pharmaceutically acceptable salt thereof, makes it possible to improve the joint function of the vertebral column.

The present invention also relates to a composition comprising nicotinamide mononucleotide (NMN), a pharmaceutically acceptable derivative thereof, or a pharmaceutically acceptable salt thereof, and at least one pharmaceutically acceptable excipient, for use thereof in the prevention and/or treatment of a back pain.

Preferably, the composition according to the invention is intended to be administered via the topical route.

Preferably the back pain is a lumbalgia or low back pain, and more preferably a chronic lumbalgia.

Advantageously, the composition according to the invention may be in the form of a gel, a solution, a water-in-oil emulsion, an oil-in-water emulsion, an oil, a cream, an ointment/salve, or a liniment.

In one preferred embodiment, the composition according to the invention is in the form of a water-in-oil emulsion or an oil-in-water emulsion; on a more preferred basis, an oil-in-water emulsion.

Advantageously, the composition according to the invention may be a pharmaceutical composition.

Advantageously, the composition according to the invention may comprise NMN, a pharmaceutically acceptable salt thereof, or a pharmaceutically acceptable derivative thereof, in an amount comprised between 0.05% and 15% by weight, preferably between 1 and 10% by weight, on a more preferred basis between 3 and 5% by weight relative to the total weight of the composition.

Advantageously, the NMN, a pharmaceutically acceptable derivative thereof, or a pharmaceutically acceptable salt thereof, is administered between 1 and 10 times per day, preferably between 1 and 5 times per day, on a more preferred basis between 1 and 3 times per day.

In one preferred embodiment, the NMN, a pharmaceutically acceptable derivative thereof, or a pharmaceutically acceptable salt thereof, is administered twice a day.

Advantageously, the composition according to the invention may also comprise at least one additional therapeutic agent as defined above for use thereof in the prevention and/or treatment of a back pain, preferably a lumbalgia or low back pain, on a more preferred basis a chronic lumbalgia.

### Definitions

In the present invention, the following terms have the following meanings.

Unless otherwise indicated, the nomenclature of substituents which are not explicitly defined in the present invention is obtained by naming the terminal portion of the functional group followed by the adjacent functional group towards the point of attachment.

“Alkyl” by itself or as part of another substituent refers to a hydrocarbyl radical having the formula CnH2n+1 in which n is a number greater than or equal to 1. In general, the alkyl groups of this invention include from 1 to 12 carbon atoms, preferably from 1 to 8 carbon atoms, more preferably from 1 to 6 carbon atoms, even more preferably from 1 to 2 carbon atoms. The alkyl groups may be linear or branched and may be substituted as indicated in the present invention. The alkyls that are suitable for the purposes of implementation of the invention may be selected from among methyl, ethyl, n-propyl, i-propyl, n-butyl, i-butyl, s-butyl and t-butyl; pentyl and its isomers such as n-pentyl and iso-pentyl; and hexyl and its isomers such as n-hexyl and iso-hexyl; heptyl and its isomers (for example n-heptyl, iso-heptyl); octyl and its isomers (for example n-octyl, iso-octyl); nonyl and its isomers (for example n-nonyl, iso-nonyl); decyl and its isomers (for example n-decyl, iso-decyl); undecyl and its isomers; dodecyl and its isomers. Preferably, the alkyl groups may be selected from among methyl, ethyl, n-propyl, i-propyl, n-butyl, i-butyl, s-butyl, t-butyl, n-pentyl, n-hexyl, n-heptyl, n-octyl, n-nonyl, and n-decyl. The saturated and branched alkyl groups may be selected, without limitation, from among isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, and 3,3-diethylhexyl. The preferred alkyl groups are the following: methyl, ethyl, n-propyl, i-propyl, n-butyl, i-butyl, s-butyl and t-butyl. Cx-Cy-alkyls refer to alkyl groups that contain from x to y carbon atoms.

When the suffix “ene” (“alkylene”) is used in conjunction with an alkyl group, it indicates that the alkyl group as defined herein has two single bonds as points of attachment to other groups. The term “alkylene” includes methylene, ethylene, methylmethylene, propylene, ethylethylene, and 1,2-dimethylethylene.

The term “alkenyl” as used herein refers to an unsaturated hydrocarbyl group, which may be linear or branched, that comprises one or more carbon-carbon double bonds. The alkenyl groups that are suitable comprise between 2 and 12 carbon atoms, preferably between 2 and 8 carbon atoms, and even more preferably between 2 and 6 carbon atoms. Examples of alkenyl groups are ethenyl, 2-propenyl, 2-butenyl, 3-butenyl, 2-pentenyl and its isomers, 2-hexenyl and its isomers, 2,4-pentadienyl and other similar groups.

The term “alkynyl”, as used herein, refers to a class of monovalent unsaturated hydrocarbyl groups, in which the unsaturation results from the presence of one or more carbon-carbon triple bond(s). The alkynyl groups generally, and preferably, have the same number of carbon atoms as described here above for the alkenyl groups. Without limitation, some examples of alkynyl groups include ethynyl, 2-propynyl, 2-butynyl, 3-butynyl, 2-pentynyl and its isomers, 2-hexynyl and its isomers, etc.

“Alkoxy” refers to an alkyl group as defined here above, which is attached to another moiety by means of an oxygen atom. Examples of alkoxy groups include the groups: methoxy, isopropoxy, ethoxy, tert-butoxy, and the like. The alkoxy groups may be optionally substituted by one or more substituent(s). The alkoxy groups included in the compounds of this invention may be optionally substituted with a solubilising group.

“Aryl”, as used herein, refers to a polyunsaturated aromatic hydrocarbyl group having a single ring (for example phenyl) or multiple aromatic rings that are fused together (for example naphthyl) or covalently bonded, which generally contains 5 to 18 atoms, preferably 5 to 12, on a more preferred basis 6 to 10, with at least one of the said rings being aromatic. The aromatic ring may optionally include one or two additional rings (cycloalkyl, heterocyclyl, or heteroaryl) fused thereto. The aryl is also intended to include partially hydrogenated derivatives of the carbocyclic systems listed herein. Examples of aryl include phenyl, biphenylyl, biphenylenyl, 5- or 6-tetralinyl; naphthalene-1- or -2-yl; 4-, 5-, 6 or 7-indenyl; 1-, 2-, 3-, 4-, or 5-acenaphthylenyl; 3-, 4-, or 5-acenaphthenyl; 1-, or 2-pentalenyl; 4-, or 5-indanyl; 5-, 6-, 7-, or 8-tetrahydronaphthyl; 1,2,3,4-tetrahydronaphthyl; 1,4-dihydronaphthyl; 1-, 2-, 3-, 4-, or 5-pyrenyl.

When at least one carbon atom in an aryl group is replaced by a heteroatom, the resulting ring is referred to herein as a “heteroaryl” ring.

“Alkylaryl” refers to an aryl group substituted by an alkyl group.

“Amino acid” refers to an alpha-amino carboxylic acid, that is to say, a molecule comprising a carboxylic acid functional group and an amino functional group in the alpha position of the carboxylic acid group, for example a proteinogenic amino acid or a non-proteinogenic amino acid.

“Proteinogenic amino acid” refers to an amino acid that is incorporated into the proteins during the translation of the messenger RNA by the ribosomes in living organisms, that is to say, Alanine (ALA), Arginine (ARG), Asparagine (ASN), Aspartate (ASP), Cysteine (CYS), Glutamate (glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR), or Valine (VAL).

“Non-proteinogenic amino acid” as used herein refers to an amino acid that is not naturally encoded or found in the genetic code of a living organism. Without limitation, some examples of non-proteinogenic amino acid are: ornithine, citrulline, argininosuccinate, homoserine, homocysteine, cysteine-sulfinic acid, 2-aminomuconic acid, δ-aminolevulinic acid, β-alanine, cystathionine, γ-aminobutyrate, dihydroxyphenylalanine (DOPA), 5-hydroxytryptophan, D-serine, ibotenic acid, α-aminobutyrate, 2-aminoisobutyrate, D-leucine, D-valine, D-alanine, and D-glutamate.

The term “cycloalkyl” as used herein refers to a cyclic alkyl group, that is to say, a monovalent, saturated or unsaturated hydrocarbyl group, having 1 or 2 ring structures. The term “cycloalkyl” includes monocyclic or bicyclic hydrocarbyl groups. The cycloalkyl groups may comprise 3 or more carbon atom(s) in the ring and generally, according to the present invention, comprise from 3 to 10, more preferably from 3 to 8 carbon atoms, and even more preferably from 3 to 6 carbon atoms. Examples of cycloalkyl groups include, but are not limited to, cyclopropyl, cyclobutyl, cyclopentyl, cyclohexyl, with cyclopropyl being particularly preferred.

The term “pharmaceutically acceptable excipient” refers to an inert carrier or support substance used as a solvent or diluent within which the active ingredient is formulated and/or administered, and which does not produce an adverse, allergic or other reaction when it is administered to an animal, preferably to a human. This includes all solvents, dispersing media, coatings, antibacterial and antifungal agents, isotonic agents, absorption retardants, and other similar ingredients. For human administration, the preparations must meet specific standards of sterility, general safety and purity, as required by the regulatory authorities, such as for example the Food and Drug Administration (FDA) in the United States of America, or the European Medicines Agency (EMA). Within the meaning of the invention, “pharmaceutically acceptable excipient” includes all pharmaceutically acceptable excipients as well as all pharmaceutically acceptable carriers, diluents and/or adjuvants.

“Halogen” or “halo” refers to fluoro, chloro, bromo or iodo. The preferred halo groups are fluoro and chloro.

“Haloalkyl” alone or in combination, refers to an alkyl radical having the meaning as defined here above, in which one or more hydrogen atom(s) are replaced by a halogen as defined here above. By way of examples of such haloalkyl radicals, the following may be cited: chloromethyl, 1-bromoethyl, fluoromethyl, difluoromethyl, trifluoromethyl, 1,1,1-trifluoroethyl, and similar radicals. ‘Cx-Cy-haloalkyl’ and ‘Cx-Cy-alkyl’ refer to alkyl groups that contain from x to y carbon atoms. The preferred haloalkyl groups are difluoromethyl and trifluoromethyl.

“Heteroalkyl” refers to an alkyl group as defined here above, in which one or more carbon atom(s) are replaced by a heteroatom selected from among oxygen, nitrogen and sulfur atoms. In the heteroalkyl groups, the heteroatoms are bonded along the alkyl chain only to carbon atoms, that is to say, each heteroatom is separated from every other heteroatom by at least one carbon atom. However, the nitrogen and sulfur heteroatoms may optionally be oxidised and the nitrogen heteroatoms may optionally be quaternised. A heteroalkyl is bonded to another group or molecule only by means of a carbon atom, that is to say, the bonding atom is not selected from the heteroatoms included in the heteroalkyl group.

The term “heteroaryl” as used herein, alone or as part of another group, refers to, but is not limited to, aromatic rings of 5 to 12 carbon atoms or ring systems containing 1 or 2 rings that are fused or covalently bonded, and generally containing 5 or 6 atoms, with at least one of the said rings being aromatic; in which one or more carbon atom(s) in one or more of these rings are replaced by oxygen, nitrogen and/or sulfur atoms, it being possible for the nitrogen and sulfur heteroatoms to optionally be oxidised and for the nitrogen heteroatoms to optionally be quaternised. These rings may be fused to an aryl, cycloalkyl, heteroaryl or heterocyclyl ring. Without limitation, some examples of such heteroaryls include: furanyl, thiophenyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyrimidyl, pyrazinyl, pyridazinyl, dioxinyl, thiazinyl, triazinyl, imidazo [2,1-b] [1,3] thiazolyl, thieno [3,2-b] furanyl, thieno [3,2-b] thiophenyl, thieno [2,3-d] [1,3] thiazolyl, thieno [2,3-d] imidazolyl, tetrazolo [1,5-a] pyridinyl, indolyl, indolizinyl, isoindolyl, benzofuranyl, isobenzofuranyl, benzothiophenyl, isobenzothiophenyl, indazolyl, benzimidazolyl, 1,3-benzoxazolyl, 1,2-benzisoxazolyl, 2,1-benzisoxazolyl, 1,3-benzothiazolyl, 1,2-benzoisothiazolyl, 2,1-benzoisothiazolyl, benzotriazolyl, 1,2,3-benzoxadiazolyl, 2,1,3-benzoxadiazolyl, 1, 2,3-benzothiadiazolyl, 2,1,3-benzothiadiazolyl, thienopyridinyl, purinyl, imidazo [1,2-a] pyridinyl, 6-oxo-pyridazin-1(6H)-yl, 2-oxopyridin-1(2H)-yl, 6-oxo-pyridazin-1(6H)-yl, 2-oxopyridin-1(2H)-yl, 1,3-benzodioxolyl, quinolinyl, isoquinolinyl, cinnolinyl, quinazolinyl, quinoxalinyl.

When at least one carbon atom in a cycloalkyl group is replaced by a heteroatom, the resulting ring is referred to herein as “heterocycloalkyl” or “heterocyclyl”.

The terms “heterocyclyl”, “heterocycloalkyl”, or “heterocyclo”, as used herein by themselves or as part of another group, refer to non-aromatic cyclic groups, either fully saturated or partially unsaturated (for example, 3 to 7 membered monocyclic, 7 to 11 membered bicyclic groups or containing a total of 3 to 10 ring atoms), which have at least one heteroatom in at least one ring containing a carbon atom. Each ring of the heterocyclic group containing a heteroatom may have 1, 2, 3, or 4 heteroatoms selected from among nitrogen, oxygen and/or sulfur atoms, where the nitrogen and sulfur heteroatoms may optionally be oxidised, and the nitrogen heteroatoms may optionally be quaternised. Any whichever of the carbon atoms of the heterocyclic group may be substituted by an oxo (for example piperidone, pyrrolidinone). The heterocyclic group may be attached to any heteroatom or carbon atom in the ring or ring system, where the valence so permits. The rings of multi-ring heterocycles may be fused, bridged and/or connected by one or more spiro atoms. Exemplary heterocyclic groups include, but are not limited to, the following groups: oxetanyl, piperidinyl, azetidinyl, 2-imidazolinyl, pyrazolidinyl, imidazolidinyl, isoxazolinyl, oxazolidinyl, isoxazolidinyl, thiazolidinyl, isothiazolidinyl, piperidinyl, 3H-indolyl, indolinyl, isoindolinyl, 2-oxopiperazinyl, piperazinyl, homopiperazinyl, 2-pyrazolinyl, 3-pyrazolinyl, tetrahydro-2H-pyranyl, 2H-pyranyl, 4H-pyranyl, 3,4-dihydro-2H-pyranyl, 3-dioxolanyl, 1,4-dioxanyl, 2,5-dioximidazolidinyl, 2-oxopiperidinyl, 2-oxopyrrolodinyl, indolinyl, tetrahydropyranyl, tetrahydrofuranyl, tetrahydroquinolinyl, tetrahydroisoquinolin-1-yl, tetrahydroisoquinolin-2-yl, tetrahydroisoquinolin-3-yl, tetrahydroisoquinolin-4-yl, thiomorpholine-4-yl, thiomorpholine-4-ylsulfoxide, thiomorpholine-4-ylsulfone, 1,3-dioxolanyl, 1,4-oxathianyl, 1H-pyrrolizinyl, tetrahydro-1,1-dioxothiophenyl, N-formylpiperazinyl, and morpholine-4-yl.

The term “precursor” as used herein also refers to pharmacologically acceptable derivatives of compounds having the formula (I) or (Ia) such as esters, of which the in vivo biotransformation product is the active drug. Precursors are characterised by increased bioavailability and are readily metabolised into active compounds in vivo. The precursors that are appropriate for the purposes of the invention include in particular carboxylic esters, in particular alkyl esters, aryl esters, acyloxyalkyl esters, and the carboxylic esters of dioxolene; ascorbic acid esters.

The term “pharmaceutically acceptable” refers to the state of being approved, or with the likelihood of being potentially approved by a regulatory body or listed in a recognised pharmacopoeia for use in animals, and more preferably in humans. It may pertain to a substance that is not biologically or otherwise undesirable; that is to say, the substance may be administered to an individual without causing adverse biological effects or deleterious interactions with one of the components of the composition within which it is contained. Preferably, a “pharmaceutically acceptable” salt or excipient refers to any salt or any excipient that is authorised by the European Pharmacopoeia (denoted as “Ph. Eur.”) and the American Pharmacopoeia (generally referred to as “United States Pharmacopeia (USP)”).

The term “active ingredient” or “therapeutic agent” refers to a molecule or a substance which when administered to a subject slows down or stops the progression, aggravation or deterioration of one or more symptom(s) of a disease or a condition; relieves the symptoms of a disease or a condition; cures a disease or a condition. According to one of these embodiments, the therapeutic ingredient is a small molecule, which is natural or synthetic. According to another embodiment, the therapeutic ingredient is a biological molecule such as, for example, an oligonucleotide, a small interfering RNA (siRNA), a microRNA (miRNA), a DNA fragment, an aptamer, an antibody and the like. “Pharmaceutically acceptable salts” include the acid addition salts and base addition salts of these said salts. Suitable acid addition salts are formed from acids that form non-toxic salts. Examples that may be cited include: acetate, adipate, aspartate, benzoate, besylate, bicarbonate/carbonate, bisulfate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, and salts of xinofoate. Suitable basic salts are formed from bases which form non-toxic salts. By way of examples, mention may be made of the salts of: aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine, and zinc. Hemisalts of acids and bases may also be formed, for example, hemisulfates and salts of chemical calcium. The preferred pharmaceutically acceptable salts are hydrochloride/chloride, bromide/hydrobromide, bisulfate/sulfate, nitrate, citrate and acetate.

Pharmaceutically acceptable salts may be prepared by one or more of the following methods:
by reacting the compound with the desired acid;
by reacting the compound with the desired base;
by removing an acid or base labile protecting group under basic or acidic conditions from a suitable precursor of the compound, or by ring opening of a suitable cyclic precursor, for example a lactone or a lactam, using the desired acid; or
by converting one salt of the compound into another by reacting the initial salt with an appropriate acid or by means of an appropriate ion exchange column.

All of these reactions are generally carried out in solution. The salt can precipitate out of the solution and may be collected by filtration or may be recovered by evaporation of the solvent. The degree of ionisation of the salt may vary from completely ionised to almost non-ionised.

The term “Solvate” is used herein to describe a molecular complex that comprises the compound of the invention and one or more pharmaceutically acceptable solvent molecules, for example, ethanol.

The term “substituent” or “substituted” indicates that a hydrogen radical on a compound or a group is replaced by any desired group which is substantially stable under the reaction conditions in an unprotected form or when it is protected by a protecting group. Examples of preferred substituents include, but are not limited to: a halogen (chloro, iodo, bromo, or fluoro); an alkyl; an alkenyl; an alkynyl, as described here above; a hydroxy; an alkoxy; a nitro; a thiol; a thioether; an imine; a cyano; an amido; a phosphonato; a phosphine; a carboxyl; a thiocarbonyl; a sulfonyl; a sulfonamide; a ketone; an aldehyde; an ester; an oxygen (—O); a haloalkyl (for example, trifluoromethyl); a cycloalkyl, which may be condensed-ring or non-condensed-ring monocyclic or polycyclic (for example, cyclopropyl, cyclobutyl, cyclopentyl or cyclohexyl); or a heterocycloalkyl, which may be condensed-ring or non-condensed-ring monocyclic or polycyclic (for example, pyrrolidinyl, piperidinyl, piperazinyl, morpholinyl, or thiazinyl); fused or unfused monocyclic or polycyclic, aryl or heteroaryl (for example, aryl, heteroaryl, pyrrolidinyl, piperidinyl, piperazinyl, morpholinyl, or thiazinyl); fused or unfused monocyclic or polycyclic (for example, aryl, heteroaryl, pyrrolidinyl, piperidinyl, piperazinyl, morpholinyl, or thiazinyl), phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazidinyl, pyridaziminyl, pyridaziminyl, benzimidazolyl, benzothiophenyl, or benzofuranyl); amino (primary, secondary or tertiary); CO₂CH₃; CONH2; OCH₂CONH₂; NH2; SO₂NH₂; OCHF₂; FC₃; OCF₃; moreover these groups may also be optionally substituted by a fused ring bridge or structure, for example —OCH₂O—. These substituents may optionally be further substituted by a substituent selected from among these groups. In certain representations, the term “substituent” or the adjective “substituted” refers to a substituent selected from the group constituted of: an alkyl, an alkenyl, an alkynyl, a cycloalkyl, a cycloalkenyl, a heterocycloalkyl, an aryl, a heteroaryl, an arylalkyl, a heteroarylalkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR′₁₃, —OC(O)R₁₃, —NR₁₃C(O)NR₁₁R₁₂, —OC(O)NR₁₁R₁₂, —NR₁₃C(O)OR₁₄, —S(O)rR13, —NR₁₃S(O)rR₁₄, —OS(O)rR₁₄, S(O)rNR₁₁R₁₂, —O, —S, and —NR₁₃, where r is 1 or 2; R₁₁ and R₁₂, for each occurrence, are independently H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl; or R₁₁ and R₁₂ taken together with the nitrogen to which they are attached are an optionally substituted heterocycloalkyl, or an optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted arylalkyl, or an optionally substituted heteroarylalkyl. In certain variants, the term “substituent” or the adjective “substituted” refers to a solubilising group.

The term “administration”, or a variant of this term (for example, “administer”), refers to providing of the active ingredient, whether alone or as part of a pharmaceutically acceptable composition, to the patient who is to receive the same in the context of treatment or prevention of a condition, a symptom, or a disease.

The terms “treating”, “curing”, and “treatment”, as used herein, are meant to include the relieving, alleviation, or ablation of a condition, or a disease and/or the symptoms associated therewith.

The terms “prevent”, “impede” and “prevention”, as used in the present invention, refer to a method that serves the purpose of: delaying, or impeding or preventing the onset of a condition, or a disease and/or the symptoms associated therewith; preventing a patient from contracting a condition or a disease; or reducing the risk of a patient's contracting a given disease or a condition.

The bonds of an asymmetric carbon may be represented herein using a solid triangle ( [symbol] ), a dotted triangle ( [symbol] ), or a zigzag line ( [symbol] ).

### DETAILED DESCRIPTION OF THE INVENTION

The object of the present invention relates to nicotinamide mononucleotide (NMN), a pharmaceutically acceptable derivative thereof, or a pharmaceutically acceptable salt thereof, for use thereof via topical administration in the prevention and/or treatment of a back pain, as well as compositions that comprise the same.

Nicotinamide adenine dinucleotide (NAD) is a coenzyme present in all living cells. NAD exists in the cell either in its oxidised form NAD+, or in its reduced form NADH. The role of NAD is that of an electron carrier that is involved in the oxidation-reduction reactions of metabolism. NAD is moreover also involved in a number of cellular processes such as adenosine diphosphate (ADP) ribosylation in the context of post-translational modifications of proteins.

NAD may be synthesised de novo by the cell from amino acids such as tryptophan or aspartate. However, such synthesis is marginal because the main pathway for NAD synthesis is the salvage pathway, by means of which the cell, and primarily the cell nucleus, recycles compounds in order to reform NAD from precursors. The precursors of NAD include niacin, nicotinamide riboside, nicotinamide mononucleotide, and nicotinamide.

NMN is one of the compounds that enable the synthesis of NAD by the salvage pathway and has the formula:

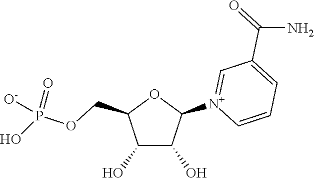 [SMILES: NC(=O)c1ccc[n+]([C@@H]2O[C@H](COP(=O)([O-])O)[C@@H](O)[C@H]2O)c1]

The present invention proposes to use NMN, the pharmaceutically acceptable derivatives thereof, or the pharmaceutically acceptable salts thereof, as well as compositions that comprise the same for preventing and/or treating lumbalgia (low back pain), that is to say back pains that are experienced in the lumbar vertebrae region. The inventors have in particular discovered that the administration of NMN via the topical route makes it possible to reduce lumbalgia (low back pain) and in particular chronic lumbalgia.

In addition, the use of NMN, which is a molecule naturally present in the body, has many advantages. In particular, NMN does not pose any tolerance problem in patients. The use of NMN and of the composition according to the invention in fact does not induce any allergies. In addition, the use of NMN and of the composition according to the invention does not induce the adverse side effects frequently encountered with conventional treatments.

In particular, NMN also does not induce any phenomenon of physical or psychological dependence, unlike analgesics that comprise morphine or opium derivatives. Furthermore, NMN also does not induce any bone fragility or vulnerability to infections as is observed with the chronic administration of cortisone or its derivatives. The use of NMN and of the composition according to the invention for preventing and/or treating back pain, preferably lumbalgia and more preferably chronic lumbalgia, is therefore safe.

The NMN and the composition according to the invention may be used for adults as also for children. NMN is indeed well tolerated by children. In the context of the invention, patients are deemed to be children if aged less than 18 years, and adults from the age of 18 onwards. Consequently, the invention is also of interest in treating back pain in children.

In one particularly preferred embodiment, the NMN is in the form of a zwitterion. The term “zwitterion” is understood to refer to a molecular chemical species that possesses electrical charges of opposite signs and situated, in general, on non-adjacent atoms of the molecule.

The pharmaceutically acceptable derivative of NMN may be selected from among dihydronicotinamide mononucleotide (denoted NMN-H), alpha-NMN; the pharmaceutically acceptable derivative of NMN may be selected from among: —a compound having the formula (I):

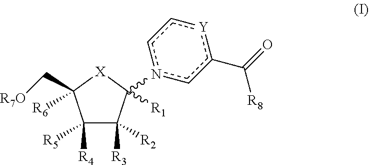

or one of the pharmaceutically acceptable: stereoisomers, salts, hydrates, solvates, or crystals thereof, in which:
X is selected from among 0, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, P(S)R₉R₁₀ and

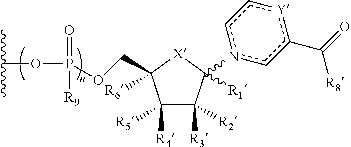

where n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂SO₂; wherein each of R₁₁ₐ is independently selected from H and a (C₁-C₆) alkyl, and R_{11b} is independently selected from OH, C₁-C₆ alkoxy, NH₂, NH(C₁-C₆alkyl) or N(C₁-C₆alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
   R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃;
[symbol] represents a single or a double bond along Y; and
[symbol] represents the alpha or beta anomer depending on the position of R₁;
   or
   a compound having the formula (Ia):

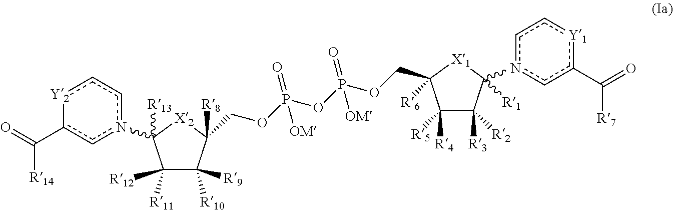

or one of the: stereoisomers, salts, hydrates, solvates, or crystals thereof, in which:
X′₁ and X′₂ are independently selected from among O, CH₂, S, Se, CHF, CF₂, and C═CH₂;
R′₁ and R′₁₃ are independently selected from among H, azido, cyano, a C1-C8 alkyl, a C1-C8 thio-alkyl, a C1-C8 heteroalkyl, and OR, wherein R is selected from H and a C1-C8 alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′₁ and Y′₂ are independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
and combinations thereof.

Within the meaning of the invention, M′ may be an internal or external counter ion.

In a first preferred embodiment, the pharmaceutically acceptable derivative is the compound having the formula (I).

In one variant of the first embodiment, X represents an oxygen.

In one variant of the first embodiment, R₁ and R₆ each independently of one another represent a hydrogen.

In one variant of the first embodiment, R₂, R₃, R₄ and R₅ each independently of one another represent a hydrogen or an OH.

In one variant of the first embodiment, Y represents a CH.

In one variant of the first embodiment, Y represents a CH₂.

In one variant of the first embodiment, R₇ represents a hydrogen.

In one variant of the first embodiment, R₇ represents P(O)(OH)₂.

In one variant of the first embodiment,

X represents an oxygen; and/or
R₁ and R₆ each independently represent a hydrogen; and/or
R₂, R₃, R₄ and R₅ each independently represent a hydrogen or R₂, R₃, R₄ and R₅ independently represent OH; and/or
Y represents a CH or a CH₂; and/or
R₇ represents P(O)R₉R₁₀, in which R₉ and R₁₀ are independently selected from among OH, OR₁₁, NHR₁₃, NR₁₃R₁₄, C₁-C₈ alkyl, C₂-C₈ alkenyl, C₂-C₈ alkynyl, C₃-C₁₀ cycloalkyl, C₅-C₁₂ aryl, C₁-C₈ aryl alkyl, C₁-C₈ alkyl aryl, C₁-C₈ heteroalkyl, C₁-C₈ heterocycloalkyl, heteroaryl, and NHCR_{A}R_{A′}C(O)R₁₂.

In one particularly preferred variant of the first embodiment, the compound of the invention is selected from among the compounds having the formula IB to IJ:

TABLE 1

| Compounds | |
|---|---|
| (Anomers) | Structure |
| I-B (alpha) | 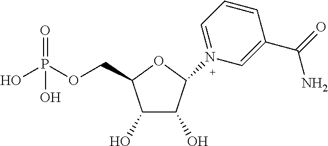 |
| I-C (beta) | 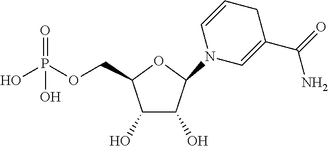 [SMILES: NC(=O)C1=CN([C@@H]2O[C@H](COP(=O)(O)O)[C@@H](O)[C@H]2O)C=CC1] |
| I-D (alpha) | 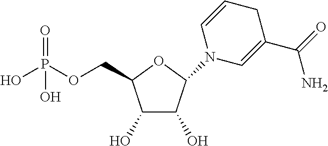 [SMILES: NC(=O)C1=CN([C@H]2O[C@H](COP(=O)(O)O)[C@@H](O)[C@H]2O)C=CC1] |
| I-E (beta) | 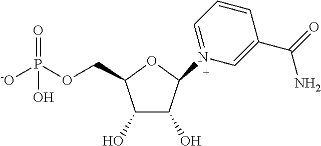 |
| I-F (alpha) | 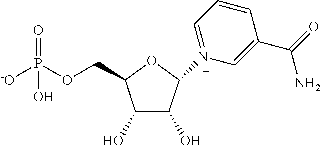 |
| I-G (beta) | 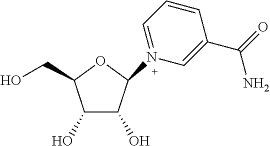 |
| I-H (alpha) | 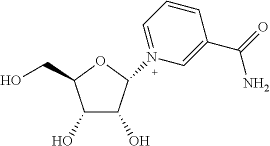 |
| I-I (beta) | 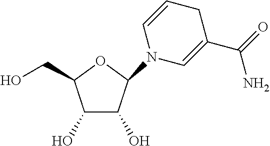 [SMILES: NC(=O)C1=CN([C@@H]2O[C@H](CO)[C@@H](O)[C@H]2O)C=CC1] |
| I-J (alpha) | 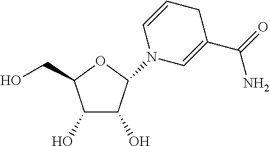 [SMILES: NC(=O)C1=CN([C@H]2O[C@H](CO)[C@@H](O)[C@H]2O)C=CC1] |

The pharmaceutically acceptable derivative of NMN may be alpha-NMN (Compounds I-B or I-F) or dihydronicotinamide mononucleotide (NMN-H) (Compounds I-D or I-C), and combinations thereof.

In a second preferred embodiment, the pharmaceutically acceptable derivative is the compound having the formula (Ia).

In one variant of the second embodiment, X′1 and X′2 each independently represent an oxygen.

In one variant of the second embodiment, R′7 and R′14 each independently represent an NH₂.

In one variant of the second embodiment, R′1 and/or R′13 each independently represent a hydrogen.

In one variant of the second embodiment, R′6 and/or R′8 each independently represent a hydrogen.

In one variant of the second embodiment, R′2, R′3, R′4, R′5, R′9, R′10, R′11, and R′12 each independently represent a hydrogen.

In one variant of the second embodiment, R′2, R′3, R′4, R′5, R′9, R′10, R′11, and R′12 each independently represent an OH.

In one variant of the second embodiment, Y′1 and Y′2 each independently represent a CH.

In one variant of the second embodiment, Y′1 and Y′2 each independently represent a CH2.

In one variant of the second embodiment, the compound according to the invention is selected from among the compounds having the formula Ia-A to Ia-I:

TABLE 2

| Compounds | |
|---|---|
| (Anomers) | Structure |
| Ia-A (beta, beta) | 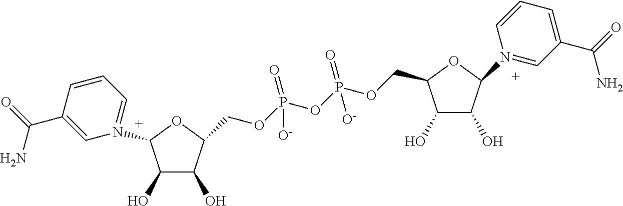 |
| Ia-B (beta, alpha) | 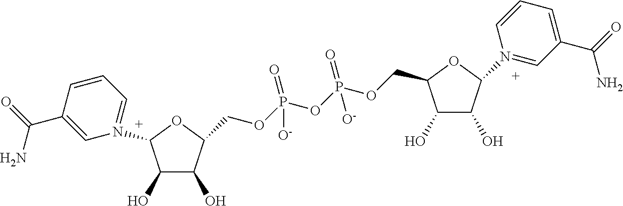 |
| Ia-C (alpha, alpha) | 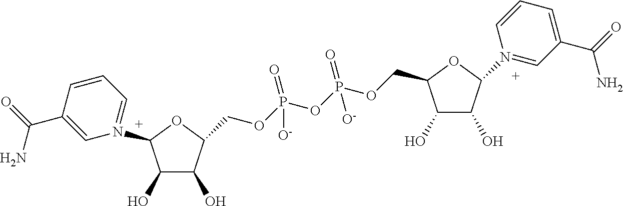 |
| Ia-D (beta, beta) | 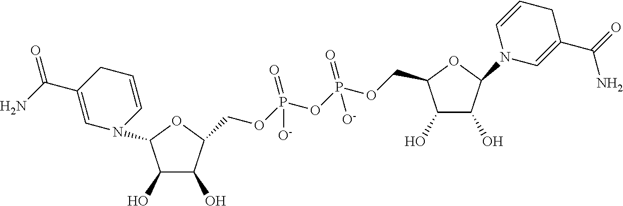 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 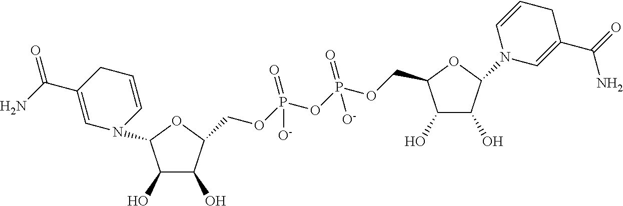 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 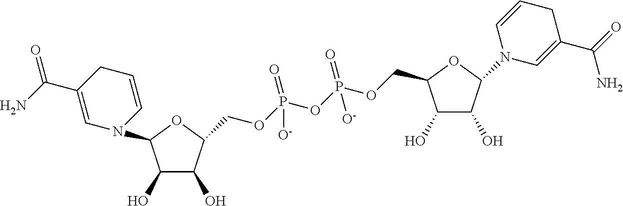 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 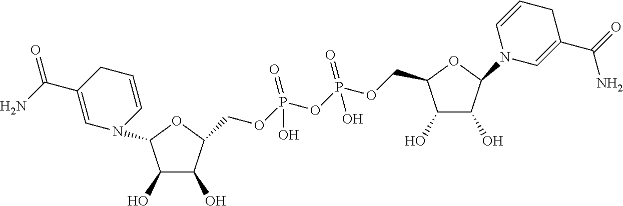 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 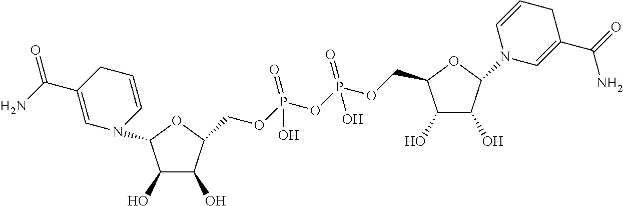 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 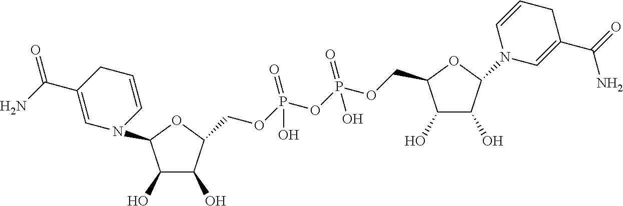 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

Preferably, the compound having the formula Ia is selected from among the compounds Ia-B, Ia-C, Ia-E, Ia-F, Ia-H and Ia-I, and combinations thereof.

NMN, a pharmaceutically acceptable derivative thereof, or a pharmaceutically acceptable salt thereof, as well as compositions that comprise the same according to the invention may be used to treat back pain, preferably lumbalgia (low back pain), on a more preferred basis chronic lumbalgia.

The use of NMN, of a pharmaceutically acceptable derivative thereof, or a pharmaceutically acceptable salt thereof, as well as compositions that comprise the same according to the invention, for treating or preventing lumbalgia, preferably chronic lumbalgia, therefore makes it possible to avoid, or at the very least to reduce, the use of conventional treatments for lumbalgia and therefore to avoid, or at the very least to reduce, the appearance of adverse side effects linked to these therapies.

Indeed, the administration of NMN, a pharmaceutically acceptable derivative thereof, or a pharmaceutically acceptable salt thereof, as well as of compositions that comprise the same, makes it possible to avoid, or at the very least to reduce, the risk of development of a lumbalgia. At the very least, it is possible to use NMN, a pharmaceutically acceptable derivative thereof, or a pharmaceutically acceptable salt thereof, as well as compositions that comprise the same, to prevent acute lumbalgia from persisting and becoming chronic.

Thus, the present invention makes it possible to offer an alternative to conventional treatments for back pain, and in particular for lumbalgia and chronic lumbalgia, and to reduce the adverse side effects of conventional therapies.

Within the meaning of the invention, lumbalgia may be classified within one of the categories M50 to M54 and G55.1, preferably within the categories M51, M54 and G55.1 of the International Classification of Diseases ICD-10.

There exist various different scales for measuring pain. Such measurement scales are for example listed in the document provided by the Haute Autorité de Santé/French National High Authority for Health (https://www.has-sante.fr/upload/docs/appication/pdf/2019-02/liste echelles douleur 2019.pdf). Among these the following noteworthy scales may be mentioned: the Visual Analogue Scale (VAS) of pain, the numerical scale, the simple verbal scale, the WOMAC index (for Western Ontario and McMaster Universities Osteoarthritis Index) and the Lequesne questionnaire. Some scales have been developed for particular categories of the population. For example, the Doloplus and Algoplus scales have been developed specifically for the elderly.

More precisely, the WOMAC score is calculated on the basis of the responses to the questions below:

Pain (5 items each evaluated from 0-100): RATE YOUR PAIN WHEN . . . .

Item 1. Walking on flat/even surface

Item 2. Ascending stairs or descending stairs

Item 3. At night while in bed

Item 4. Rising from sitting or sitting down

Item 5. Standing upright

Stiffness (2 items each rated from 0-100): RATE THE STIFFNESS IN YOUR BACK WHEN . . . .

Item #1. Rising from bed in the morning

Item 2. Rising/moving after sitting, lying or resting during the day

Physical Function (17 items each rated from 0-100): RATE YOUR DIFFICULTY WHEN . . . .

Item 1. Descending stairs

Item 2. Ascending stairs

Item 3. Rising from sitting

Item 4. Standing upright

Item 5. Bending to floor

Item 6. Walking on even floor

Item 7. Getting in and out of car

Item 8. Going shopping

Item 9. Putting on tights or socks

Item 10. Rising from bed

Item 11. Taking off tights or socks

Item 12. Lying in bed

Item 13. Getting in or out of bath

Item 14. Sitting

Item 15. Getting on and off toilet

Item 16. Doing light domestic chores/tasks (eg cooking, dusting)

Item 17. Doing heavy domestic chores/tasks (eg moving furniture)

The total score corresponds to the average of the 24 items. The same is true for the score for each area of assessment.

As for the Lequesne score, it varies from 0 to 22: the higher the score, the more extreme or even intolerable the impairment. From 8 to 10, the impairment is qualified as significant and for an index score greater than or equal to 10, the impairment is qualified as very significant.

In particular, NMN, a pharmaceutically acceptable derivative thereof, or a pharmaceutically acceptable salt thereof, or compositions that comprise the same may be used to improve the function, stiffness and pain parameters of the WOMAC index.

NMN, a derivative thereof or a pharmaceutically acceptable salt thereof, or compositions that comprise the same may be used to reduce back pain, spinal joint stiffness and/or improve spinal joint function.

The term “joint function” is understood to refer to the movements of flexion, extension, lateral flexion and rotation of the vertebral column that are made possible by the vertebrae, in particular the lumbar vertebrae.

Use

According to the present invention, NMN, the pharmaceutically acceptable derivatives thereof, or the pharmaceutically acceptable salts thereof, as well as compositions that comprise the same, are used to prevent and/or treat back pain.

Within the meaning of the invention, the back pain may be due to one of the pathologies selected from among: injury to a muscle, injury to a ligament, injury to a tendon, degeneration of the intervertebral discs, a herniated disc, a pain that is gynecological in origin, spondylolisthesis, arthritis, osteoarthritis, osteoporosis of the vertebral column (or spine), osteoporosis-related fracture, an abdominal aortic aneurysm, a tumour, an infection, an inflammation, facet joint injuries, intervertebral disc injuries, regional or global spinal (in)stability-related [spinal statics] disorders, muscular contraction in the vertebrae, muscle tearing, spinal deformity, or combinations thereof.

Spondylolisthesis refers to a condition of the human skeleton, characterised by one vertebra slipping forward and onto the vertebra located below it (antepondylolisthesis) or behind (retrolisthesis).

The spinal deformity may be scoliosis, kyphosis, lordosis or spina bifida.

The term “injury” (lesion) is understood to refer to any alteration of the anatomical or histological characteristics of an organ, a tissue or a cell, whether resulting from a pathological or traumatic condition.

According to the invention, the back pain may be classified within one of the categories of the International Classification of Diseases ICD-10, preferably within the categories M40 to M43; M46 to M54 and G55.

According to the invention, the back pain is not ankylosing spondylitis.

Preferably the back pain is a cervicalgia (neck pain) or a lumbalgia (low back pain), and on a more preferred basis, a chronic lumbalgia. Lumbalgia may be defined as a pain felt in the lumbar vertebrae. Humans have five lumbar vertebrae numbered from L1 to L5. The lumbar vertebrae are located in the caudal part of the vertebral column, more precisely between the sacrum and the thoracic vertebrae. The role of the lumbar vertebrae is to enable flexion and extension movements of the vertebral column, as well as, to a lesser extent, lateral flexion and rotation movements. They also support a large portion of the weight of the body. Given their anatomical role and the constant stress they undergo, lumbalgia is one of the most common back pain pathologies.

On a more preferred basis, the back pain, preferably the cervicalgia (neck pain) or lumbalgia (low back pain), and more preferably chronic lumbalgia, are due to an inflammation, a muscle spasm (muscle contraction), muscle tear, a ligament injury, a tendon injury, or combinations thereof.

The inflammation, muscle spasm/contracture, muscle tear, ligament or tendon injury may result from an involuntary movement, a strain or effort, or a repeated movement.

NMN, a derivative thereof, or a salt thereof, as well as compositions that comprise the same according to the invention may in fact be used to relieve lumbalgia (low back pain), and in particular chronic lumbalgia, without resorting to the use of conventional treatments.

In particular, NMN, a pharmaceutically acceptable derivative thereof, or a pharmaceutically acceptable salt thereof, as well as compositions that comprise the same may be used in the treatment and/or prevention of lumbalgia in mammals, preferably humans.

Thus, the use of NMN, of a pharmaceutically acceptable derivative thereof, or a pharmaceutically acceptable salt thereof, as well as compositions that comprise the same, makes it possible to avoid resorting to the use of conventional therapies or at the very least to reducing the dosage and/or the frequency of administration thereof, and therefore the adverse side effects thereof.

Mode of Administration and Galenic Form

According to the invention, NMN, a pharmaceutically acceptable derivative thereof, or a pharmaceutically acceptable salt thereof, and compositions that comprise the same are intended to be administered via the topical route. The term “topical route” is understood to refer to the form of administration of a composition or a substance at a site or on an external surface of the body, such as the skin or the mucous membranes.

The galenic or pharmaceutical dosage forms that are suitable for implementing the invention are a gel, a solution, a water-in-oil emulsion, an oil-in-water emulsion, an oil, a cream, an ointment/salve, or a liniment.

The term “solution” is understood to refer to a liquid galenic form used for the administration of at least one active ingredient that is obtained by dissolving the various ingredients in a liquid phase so as to form only one homogeneous phase.

The term “emulsion” is understood to refer to a heterogeneous mixture of two immiscible liquid substances, one thereof being dispersed in the form of small droplets in the other. These are two liquids which do not mix spontaneously (immiscible), like water and oil, but which upon undergoing specific operations (agitation, mixing, addition of some active ingredients) go on to adopt an appearance that is macroscopically homogeneous, but microscopically heterogeneous. One of the substances will therefore be dispersed in the second substance in the form of droplets. The mixture is able to remain stable thanks to a third ingredient referred to as emulsifier (speed or kinetics of evolution of the mixture is almost zero). A “water-in-oil emulsion”, denoted “water/oil”, is composed of an aqueous phase dispersed in an oily phase. An “oil-in-water emulsion”, denoted “oil/water”, is composed of an oily phase dispersed in an aqueous phase.

The term “cream” is understood to refer to a semi-solid preparation intended to be administered for topical use.

The term “ointment/salve” is understood to refer to a semi-solid preparation intended to be applied over the skin.

The term “liniment” is understood to refer to a liquid pharmaceutical form, conventionally comprising fatty substances such as oils, intended to be used by rubbing (friction) action.

The term “gel” is understood to refer to a solid material, possibly ductile, consisting of a three-dimensional network of macromolecules surrounded by liquid. A composition in the form of a gel penetrates well and rapidly into the skin and also serves to provide an anesthetic sensation of freshness. In one embodiment, the gel may be a hydrophobic gel or a hydrophilic gel. Advantageously, the gel is a hydrophilic gel.

In one preferred embodiment, the composition according to the invention is in the form of a water-in-oil emulsion or an oil-in-water emulsion, on a more preferred basis, an oil-in-water emulsion.

Indeed, NMN and its derivatives are very hydrophilic and therefore dissolve better in aqueous phases.

Advantageously, the composition according to the invention may comprise NMN, a pharmaceutically acceptable salt thereof, or a pharmaceutically acceptable derivative thereof, in an amount comprised between 0.05% and 15% by weight, preferably between 1 and 10% by weight, on a more preferred basis between 3 and 5% by weight relative to the total weight of the composition.

Advantageously, the NMN, a pharmaceutically acceptable derivative thereof, or a pharmaceutically acceptable salt thereof, is administered between 1 and 10 times per day, preferably between 1 and 5 times per day, on a more preferred basis between 1 and 3 times per day.

In one particularly preferred embodiment, the NMN, a pharmaceutically acceptable derivative thereof, or a pharmaceutically acceptable salt thereof, or the composition comprising the same is administered twice a day.

Therapeutic Combinations

NMN, a pharmaceutically acceptable derivative thereof, a pharmaceutically acceptable salt thereof, as well as compositions that comprise the same may also be used in combination with at least one other therapeutic agent, in particular the therapeutic agents conventionally used to relieve back pain, preferably lumbalgia (low back pain), on a more preferred basis chronic lumbalgia.

Among the therapeutic agents that may be combined with the invention, mention may be made of an analgesic, a non-steroidal anti-inflammatory drug, cortisone, a cortisone derivative, a muscle relaxant, and combinations thereof.

Advantageously, the analgesic may be selected from among paracetamol, nefopam, ketanin, tetrahydrocannabinol, cannabinoids, aspirin, methyl salicylate, diflunisal, salicylamide, codeine, alfentanil, carfentanil, dihydrocodeine, codeinone, tramadol, morphine, morphinone, buprenorphine, fentanyl, acetyl fentanyl, remifentanil, sufentanil, heroin, hydromorphone, nalbuphine, oxycodone, hydroxycodone, oxymorphone, laudanum, methadone, pethidine, dextropropoxyphene, endorphin, tapentadol, thebaine, vicodin, and combinations thereof.

Advantageously, the non-steroidal anti-inflammatory drug may be selected from among ibuprofen, ketoprofen, naproxen, ketorolac, alminoprofen, aceclofenac, mefenamic acid, niflumic acid, tiaprofenic acid, celecoxib, rofecoxib, valdecoxib, parecoxib, dexketoprofen, diclofenac, etodolac, etoricoxib, fenoprofen, flurbiprofen, indomethacin, meloxicam, nabumetone, piroxicam, sulindac, tenoxicam, nimesulide, and combinations thereof.

The cortisone derivative may be selected from among betamethasone, ciprofloxacin, cortivazol, dexamethasone, fludrocortisone, methylprednisolone, prednisolone and triamcinolone, and combinations thereof.

Advantageously, the muscle relaxant may be selected from among centrally acting muscle relaxants, peripherally acting muscle relaxants, direct acting muscle relaxants, and combinations thereof.

Advantageously, the centrally acting muscle relaxant may be selected from among baclofen, mephenesin, tetrazepam, thiocolchoside, tizanidine, carbamic acid esters, and combinations thereof.

The carbamic esters may be methocarbamol.

Advantageously, the peripherally acting muscle relaxants may be selected from among blockers (inhibitors) of acetylcholine release at the neuromuscular junction such as botulinum toxin type A and botulinum toxin type B, voltage gated sodium channel blockers such as conotoxins and huwentoxins, voltage-gated calcium channel blockers such as dihydropyridines, of muscle nicotinic acetylcholine receptor blockers such as curares or conotoxins.

Advantageously, the direct-acting muscle relaxant is a ryanodine receptor blocker such as dantrolene.

By way of non-limiting examples of muscle relaxants that may be used in combination with the invention, mention may in particular be made of baclofen, quinine, mephenesin, tizanidine, tetrazepam, thiocolchicoside, acetyl hexapeptide-8, p-conotoxin CnIIIc (mu-conotoxin CnIIIc), dipeptide diaminobutyroyl benzylamide diacetate as well as locally used botulinum toxin, and combinations thereof.

Acetyl hexapeptide-8 is also referred to as argireline and is registered under CAS number: 616204-22-9. Its action mimics the action of botulinum toxin. The μ-conotoxin CnIIIc (or mu-conotoxin CnIIIc) enables the blocking of the Nav1.4 sodium channels. μ-conotoxin CnIIIc (or mu-conotoxin CnIIIc) is registered under CAS number: 936616-33-0 and under UNIPROT number I1SB07. Dipeptide diaminobutyroyl benzylamide diacetate is registered under CAS number: 823202-99-9. It is used to reduce muscle contraction. Argireline, p-conotoxin CnIIIc, and dipeptide diaminobutyroyl benzylamide diacetate are preferably intended to be administered via the topical route.

The at least one other additional therapeutic agent may be administered via either topical or oral routes, or by injection. More precisely, the at least one other therapeutic agent may be administered by the route by which it is conventionally administered.

The at least one other therapeutic agent may also be administered concomitantly with, or at different times from the NMN, a pharmaceutically acceptable salt thereof, or a pharmaceutically acceptable derivative thereof, or from the composition according to the invention.

The additional therapeutic agent may be administered in order to enhance the action of the NMN, a pharmaceutically acceptable salt thereof, a pharmaceutically acceptable derivative thereof, or the said compositions.

The composition and the compounds according to the invention may be administered simultaneously, separately or sequentially with the at least one additional therapeutic agent. The term “simultaneously” is understood to indicate that two agents are administered at the same time. The term “separately”, is understood to indicate that the time interval between the administration of the first agent and that of the second is significant and at least one hour. The term “sequentially” is understood to indicate that the two agents are administered one after the other within a timeframe such that they are both available to act therapeutically over the same time period. The optimum time interval between administration of the two agents will vary depending on the precise nature of the method of administration of the compounds or compositions of the invention.

Compositions

The compositions according to the invention may comprise nicotinamide mononucleotide, a pharmaceutically acceptable derivative thereof, or a pharmaceutically acceptable salt thereof, and at least one pharmaceutically acceptable excipient for the topical administration thereof for use in the prevention and/or the treatment of a back pain, preferably a lumbalgia (low back pain), on a more preferred basis a chronic lumbalgia.

Such compositions are of utility value in particular in relieving lumbalgia, preferably chronic lumbalgia.

In the context of the present invention, an “excipient” refers to any substance other than the NMN that is in the composition and has no therapeutic effect. The excipient does not interact chemically with the NMN or any other additional therapeutic agent.

The excipient may be selected from among a bulking agent, a lubricant, a flavouring agent, a colouring agent, an emulsifier, a compression agent, a diluent, a preservative, a gelling agent, a plasticiser, a surfactant, or combinations thereof. A person skilled in the art would know how to determine the excipient to be selected based on the galenic form that they would have selected.

The composition according to the invention may be a pharmaceutical composition. In this case, the excipient is a pharmaceutically acceptable excipient.

In the context of the invention, a “pharmaceutically acceptable” salt or excipient refers to any salt or excipient that is authorised by the European Pharmacopoeia (denoted “Ph. Eur.”) and the American Pharmacopoeia (typically denoted by “United States Pharmacopeia (USP)”).

In one preferred embodiment, the composition according to the invention may further comprise at least one other additional therapeutic agent as defined above for use thereof in the prevention and/or treatment of a back pain, preferably lumbalgia (low back pain) and more preferably chronic lumbalgia. On a more preferred basis, the at least one therapeutic agent may be an analgesic, a non-steroidal anti-inflammatory drug or a muscle relaxant.

### Method for Preparing the Compounds Having the Formula (I) and (Ia)

The compounds having the formula (I) or the formula (Ia) may be prepared according to any method well known to the person skilled in the art.

### Compound Preparation Method for Preparing the Compounds Having the Formula (I)

The compounds having the formula (I) may in particular be prepared according to the methods described in the international patent application WO 2017/024255A1, and the U.S. Pat. No. 10,611,790 B2, as well as according to the method described below.

In particular, the compounds having the formula (I) disclosed herein may be prepared as described here below from the substrates A-E. It is to be understood by the person skilled in the art that these reaction schemes are by no means intended to be limiting and that variations thereto may be made without departing in spirit and scope from the present invention.

According to one embodiment, the invention relates to a compound preparation method for preparing the compounds having the formula (I) as described here above.

The method involves, in a first step, the mono-phosphorylation of a compound having the formula (A), in the presence of phosphoryl chloride and a trialkyl phosphate, so as to thereby yield the phosphorodichloridate having the formula (B),

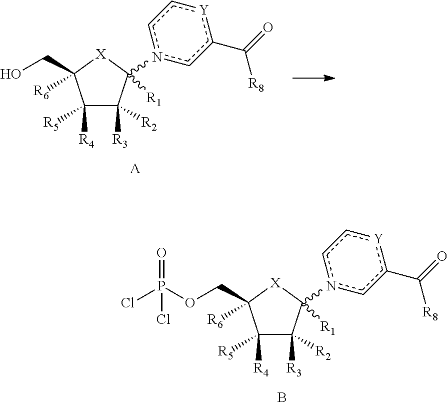

in which X, R₁, R₂, R₃, R₄, R₅, R₆, R₈, Y, [symbol] and [symbol] are as defined here above for the compounds having the formula (I).

In a second step, the phosphorodichloridate having the formula (B) is hydrolysed so as to thereby yield the phosphate having the formula (C),

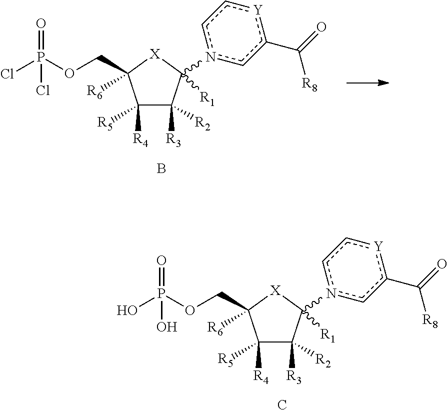

in which X, R₁, R₂, R₃, R₄, R₅, R₆, R₈, Y, [symbol] and [symbol] are as defined here above for the compounds having the formula (I).

According to one embodiment, the compound having the formula (A) is synthesised by means of various methods known to the person skilled in the art.

According to one embodiment, the compound having the formula (A) is synthesised by reaction of the pentose having the formula (D) with a nitrogenous derivative having the formula (E), in which R, R₂, R₃, R₄, R₅, R₆, R₇, Y, are as described here above for the compounds having the formula I, so as to thereby yield the compound having the formula (A-1) which is then selectively deprotected in order to give the compound having the formula (A),

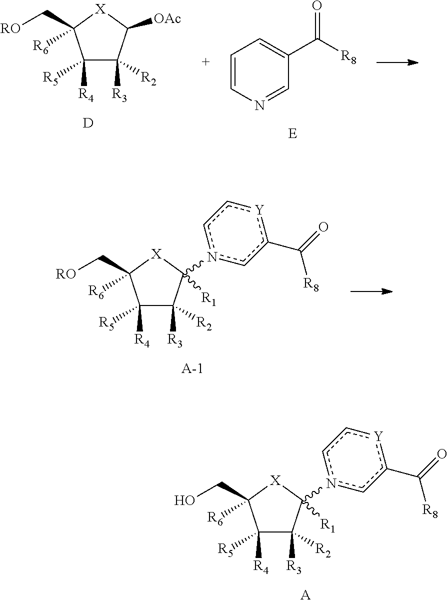

in which X, R₁, R₂, R₃, R₄, R₅, R₆, R₈, Y, [symbol] and [symbol] are as defined here above for the compounds having the formula (I).

According to one embodiment, R is a suitable protecting group known to the person skilled in the art. In one embodiment, the protecting group is selected from among triarylmethyls and/or silyls. Without limitation, some examples of triarylmethyl include trityl, monomethoxytrityl, 4,4′-dimethoxytrityl, and 4,4′,4″-trimethoxytrityl groups. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one embodiment, any hydroxyl group attached to the pentose is protected by an appropriate protecting group known to the person skilled in the art.

The selection and exchanging of the protecting groups is well within the scope of knowledge and expertise of the person skilled in the art. The protecting groups may also be removed by methods well known to the person skilled in the art, for example, with an acid (for example, an inorganic or organic acid), a base or a fluoride source.

In one preferred embodiment, the nitrogenous derivative having the formula (E) is coupled to the pentose having the formula (D) by a reaction in the presence of a Lewis acid so as to thereby yield the compound having the formula (A-1). Without limitation, some examples of Lewis acids include Trimethylsilyl Trifluoromethanesulfonate (TMSOTf), BF₃·OEt₂, TiCl₄ and FeCl₃.

In one embodiment, the method of the present invention additionally also comprises a reduction step of reducing the compound having the formula (A) by various methods well known to the person skilled in the art, so as to thereby yield the compound having the formula (A′) in which is CH₂, and R₁, R₂, R₃, R₄, R₅, R₆, R₈, Y, [symbol] and [symbol] are as defined here above for the compounds having the formula (I).

In one particular embodiment, the present invention relates to a compound preparation method for preparing the compounds having the formula I-A, I-C, I-E, I-G.

In a first step, the nicotinamide having the formula E is coupled to the ribose tetraacetate having the formula D by a coupling reaction in the presence of a Lewis acid, so as to thereby yield the compound having the formula A-1:

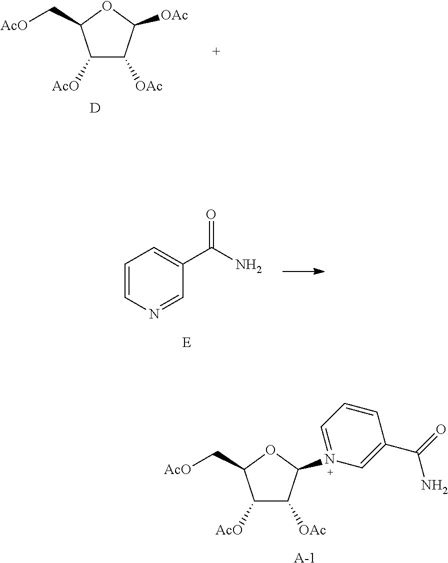

In a second step, an ammoniacal treatment of the compound having the formula A-1 is carried out, so as to thereby yield the compound having the formula I-A:

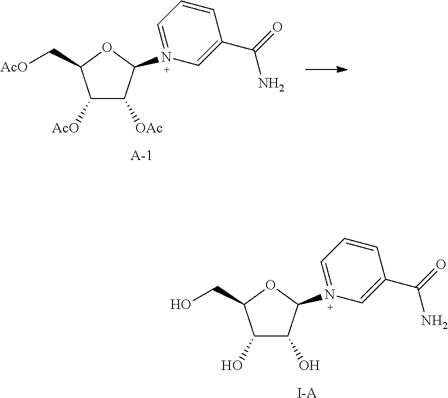

In a third step, the mono-phosphorylation of the compound having the formula I-A, in the presence of phosphoryl chloride and a trialkyl phosphate, thereby yields the phosphorodichloridate having the formula I-A′:

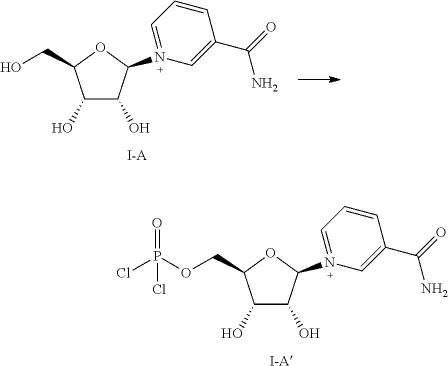

In a fourth step, the phosphorodichloridate having the formula B is hydrolysed so as to thereby yield the compound having the formula I-C:

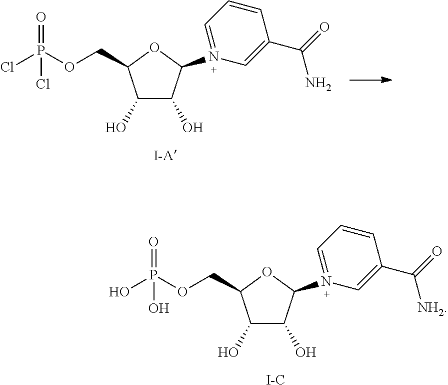

In one embodiment, a reduction step of reducing the compound having the formula I-A is carried out, so as to thereby yield the compound having the formula I-E.

The compound having the formula I-E is then mono-phosphorylated as described in the fourth step and hydrolysed so as to thereby yield the compound having the formula I-G.

According to one embodiment, the compounds having the formula (I) are selected from compounds I-A to I-H in the table below:

TABLE 1

| Compounds | |
|---|---|
| (Anomers) | Structure |
| I-A (beta) | 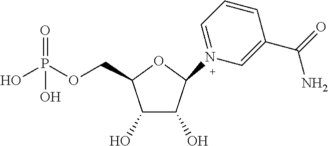 |
| I-B (alpha) | 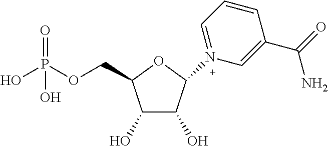 |
| I-C (beta) | 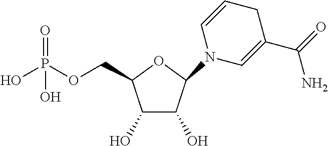 [SMILES: NC(=O)C1=CN([C@@H]2O[C@H](COP(=O)(O)O)[C@@H](O)[C@H]2O)C=CC1] |
| I-D (alpha) | 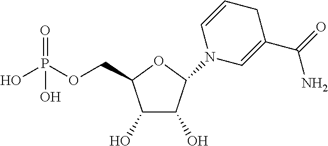 [SMILES: NC(=O)C1=CN([C@H]2O[C@H](COP(=O)(O)O)[C@@H](O)[C@H]2O)C=CC1] |
| I-E (beta) | 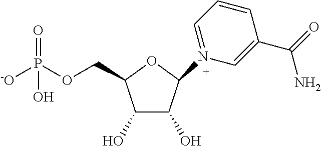 |
| I-F (alpha) | 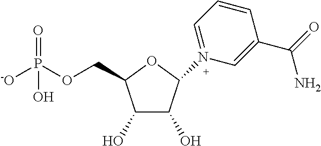 |
| I-G (beta) | 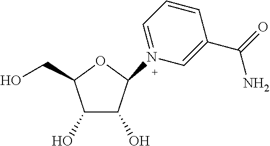 |
| I-H (alpha) | 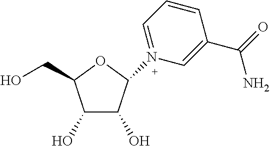 |
| I-I (beta) | 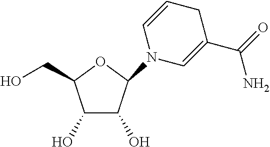 [SMILES: NC(=O)C1=CN([C@@H]2O[C@H](CO)[C@@H](O)[C@H]2O)C=CC1] |
| I-J (alpha) | 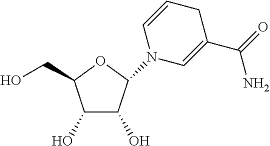 [SMILES: NC(=O)C1=CN([C@H]2O[C@H](CO)[C@@H](O)[C@H]2O)C=CC1] |

Preferably, the compound having the formula (I) is selected from among: Compound I-A, Compound I-B, Compound I-C, Compound I-D, Compound I-E, Compound I-F, Compound I-G, Compound I-H, Compound I-I, Compound I-J; preferably Compound I-C, Compound I-D or Compound I-F, and combinations thereof. On a more preferred basis, the compound having the formula (I) is selected from among Compound I-B, Compound I-C, Compound I-D, Compound I-F, and combinations thereof.

Derivative Preparation Method for Preparing the Derivatives Having the Formula (Ia)

In particular, the compounds having the formula Ia presented herein may be prepared as described here below from the substrates X-XIII. It is to be understood by the average person skilled in the art that these diagrams are by no means intended to be limiting and that variations thereto in terms of the detail may be made without departing in spirit and scope from the present invention.

According to one embodiment, the invention relates to a compound preparation method for preparing the compound having the formula I described here above.

The method consists first of all in mono-phosphorylating a compound having the formula X, in the presence of phosphoryl chloride in a trialkyl phosphate, in order to obtain the compound phosphorodichloridate XI,

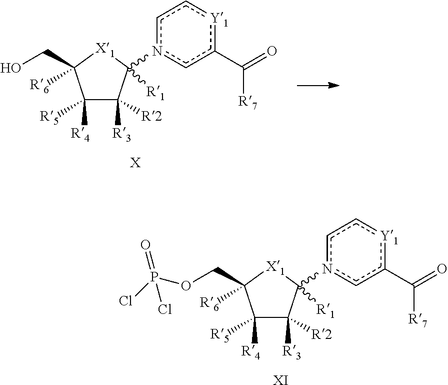

in which X′₁, R′₁, R′₂, R′₃, R′₄, R′₅, R′₆, R′₇, Y′₁, [symbol] and [symbol] are as defined here above.

In a second step, the hydrolysis of the phosphorodichloridate XI obtained in the first step gives the phosphate compound having the formula XII,

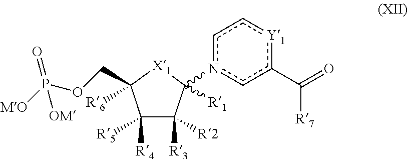

in which X′₁, R′₁, R′₂, R′₃, R′₄, R′₅, R′₆, R′₇, Y′₁, M′, [symbol] and [symbol] are as defined here above.

The phosphate compound having the formula XII obtained in the second step is then reacted with a phosphorodichloridate compound having the formula XIII obtained as described in the first step,

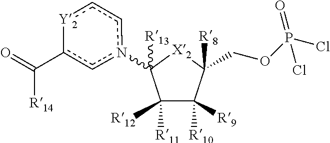

in which X′₂, R′₈, R′₉, R′₁₀, R′₁₁, R′₁₂, R′₁₃, R′₁₄, Y′₂, [symbol] and [symbol] are as described herein for formula Ia, in order to give the compound having the formula Ia as described herein.

According to one embodiment, the method further comprises a reduction step of reducing the compound having the formula Ia, using various methods known to specialists, in order to give the compound having the formula Ia, where Y′₁ and Y′₂ are identical and each represent CH₂, and where X′₁, X′₂, R′₁, R′₂, R′₃, R′₄, R′₅, R's, R′₇, R′₈, R′₉, R′₁₀, R′₁₁, R′₁₂, R′₁₃, R′₁₄, Y′₁, Y′₂, and [symbol] , [symbol] are as described herein for formula Ia.

In one variant, R is a suitable protecting group known to the person skilled in the art. Triarylmethyl and/or silyl groups are examples of suitable protecting groups. Without limitation, some examples of triarylmethyl include trityl, monomethoxytrityl, 4,4′-dimethoxytrityl, and 4,4′,4″-trimethoxytrityl. Without limitation, some examples of silyl groups include trimethylsilyl, tert-butyldimethylsilyl, triisopropylsilyl, tert-butyldiphenylsilyl, tri-iso-propylsilyloxymethyl, and [2-(trimethylsilyl)ethoxy]methyl.

According to one representation, any hydroxy group attached to the pentose ring is protected by a suitable protecting group known to the person skilled in the art.

The selection and exchanging of the protecting groups is well within the scope of knowledge and expertise of the person skilled in the art. Any protecting group may also be removed by methods known in the art, for example, with an acid (for example, an inorganic or organic acid), a base or a fluoride source.

According to one preferred embodiment, the nitrogen compounds having the formula XV are added to the pentose XIV by a coupling reaction in the presence of a Lewis acid in order to give the compound having the formula X-1. Without limitation, some examples of suitable Lewis acids include Trimethylsilyl Trifluoromethanesulfonate (TMSOTf), BF₃·OEt₂, TiCl₄ and FeCl₃.

According to one specific embodiment, the invention relates to a compound preparation method for preparing the compound having the formula VIII,

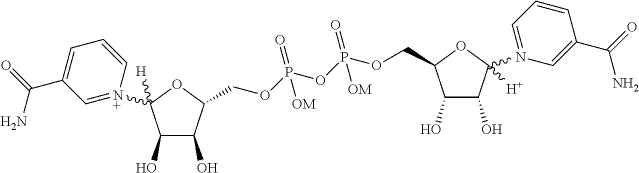

or the pharmaceutically acceptable salts and/or solvates thereof.

In a first step, the nicotinamide having the formula XV is added to the ribose tetraacetate XIV, by a coupling reaction in the presence of a Lewis acid, in order to give the compound having the formula X-1:

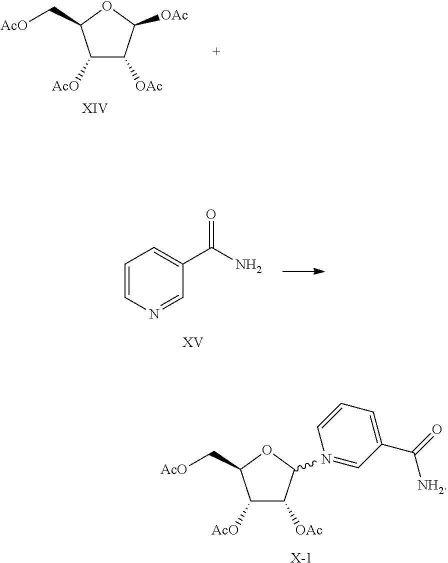

In a second step, an ammoniacal treatment of the compound having the formula X-1 gives the compound having the formula X:

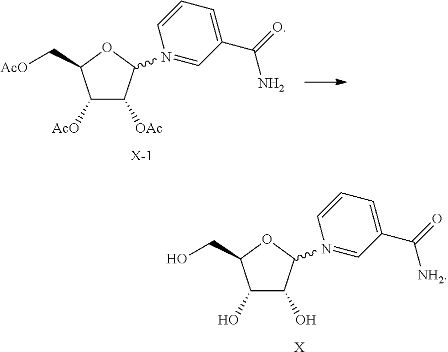

In a third step, the mono-phosphorylation of a compound having the formula X, in the presence of phosphoryl chloride in a trialkyl phosphate, gives the compound phosphorodichloridate XI:

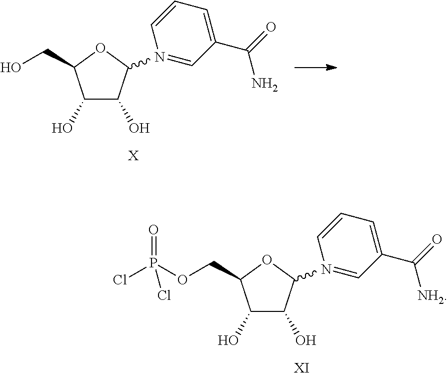

In a fourth step, the phosphorodichloridate compound XI obtained in the third step is partially hydrolysed in order to give the phosphate compound having the formula XII:

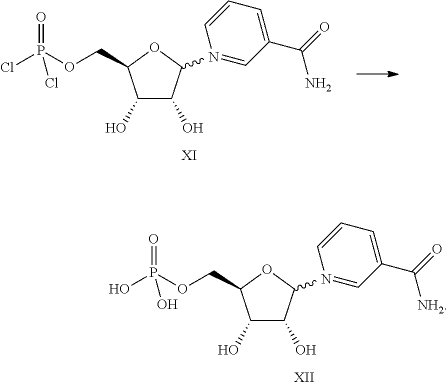

In a fifth step, the phosphate compound having the formula XII obtained in the fourth step is then reacted with the phosphorodichloridate compound having the formula XI obtained as described in the third step, in order to obtain the compound having the formula VIII.

According to another specific implementation embodiment, the invention relates to a compound preparation method for preparing the compound having the formula IX,

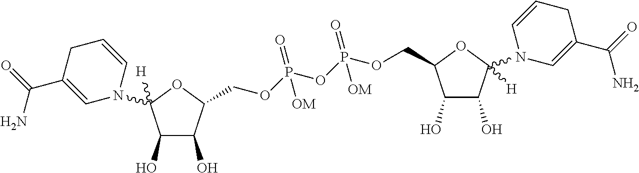

or the pharmaceutically acceptable salts and/or solvates thereof.

According to one variant, the compound having the formula IX is obtained from the compound having the formula VIII, which is synthesised beforehand as described here above.

In this embodiment, the compound having the formula IX is obtained by reducing the compound having the formula VIII, using a suitable reducing agent known to the specialised person skilled in the art, in order to give the compound having the formula IX.

According to one embodiment, the preferred compounds of the invention are the compounds Ia-A to Ia-I of Table 2:

TABLE 2

| Compounds | |
|---|---|
| (Anomers) | Structure |
| Ia-A (beta, beta) | 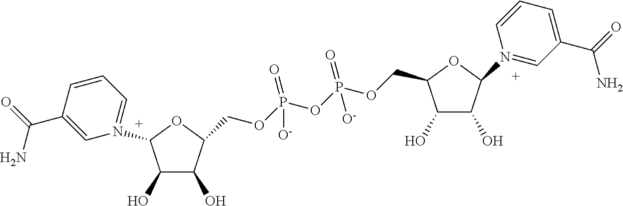 |
| Ia-B (beta, alpha) | 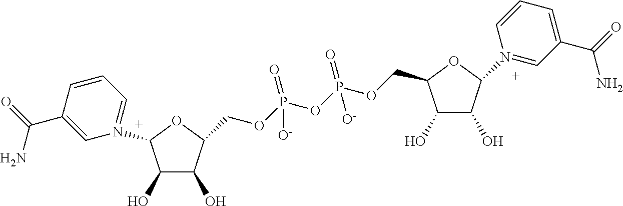 |
| Ia-C (alpha, alpha) | 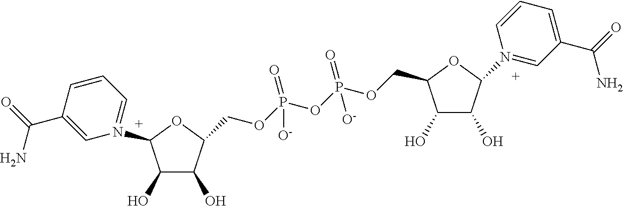 |
| Ia-D (beta, beta) | 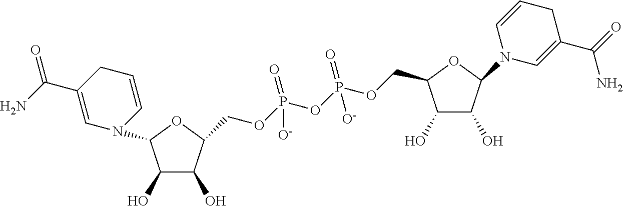 [SMILES: NC(=O)C1=CN([C@@H]2O[C@H](COP(=O)([O-])OP(=O)([O-])OC[C@H]3O[C@@H](N4C=CCC(C(N)=O)=C4)[C@H](O)[C@@H]3O)[C@@H](O)[C@H]2O)C=CC1] |
| Ia-E (beta, alpha) | 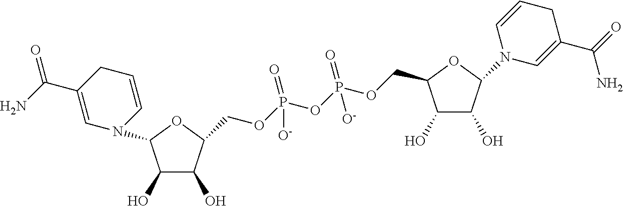 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-F (alpha, alpha) | 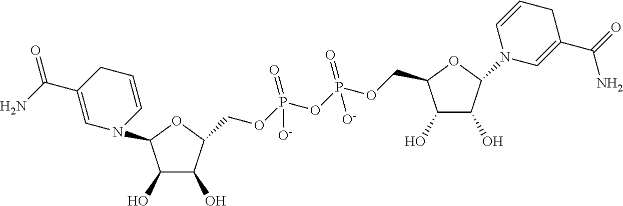 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| Ia-G (beta, beta) | 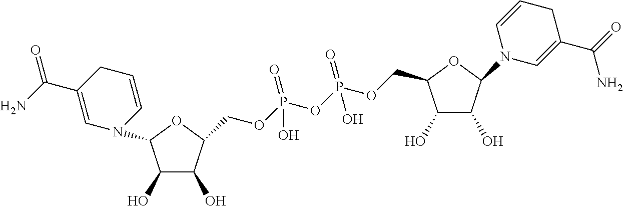 [SMILES: NC(=O)C1=CN([C@@H]2O[C@H](COP(=O)(O)OP(=O)(O)OC[C@H]3O[C@@H](N4C=CCC(C(N)=O)=C4)[C@H](O)[C@@H]3O)[C@@H](O)[C@H]2O)C=CC1] |
| Ia-H (beta, alpha) | 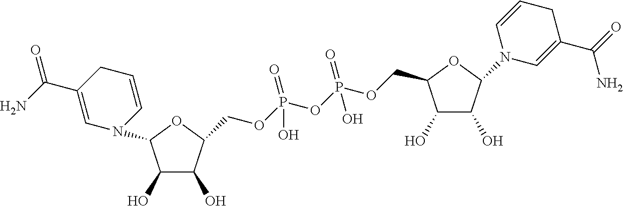 [SMILES: NC(=O)C1=CN([C@@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |
| Ia-I (alpha, alpha) | 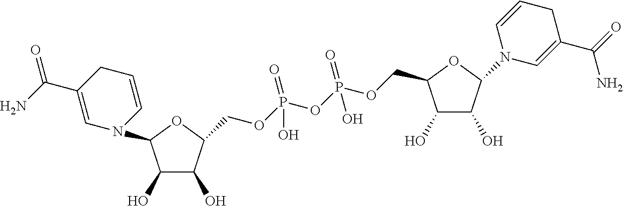 [SMILES: NC(=O)C1=CN([C@H]2O[C@H](COP(=O)(O)OP(=O)(O)OC[C@H]3O[C@H](N4C=CCC(C(N)=O)=C4)[C@H](O)[C@@H]3O)[C@@H](O)[C@H]2O)C=CC1] |

Preferably, the compound having the formula (Ia) is selected from among the compound having the formula Ia-B, the compound having the formula Ia-C, the compound having the formula Ia-E, the compound having the formula Ia-F, the compound having the formula Ia-H, the compound having the formula Ia-I, and the compound having the formula Ia-G as well as combinations thereof.

### FIGURES

FIG. 1 is a graph showing the evolution of intensity of the pain linked to lumbalgia (low back pain) over 10 days as measured by the visual analogue scale (VAS).

FIG. 2 is a graph showing the evolution of the WOMAC score and its different areas of assessment over 10 days.

FIG. 3 is a graph showing the evolution of the Lequesne score and its different categories over 10 days.

### EXAMPLES

In the remainder of this description, the examples provided are intended byway of illustration of the present invention and are in no way intended to limit the scope thereof.

### Example 1: Synthesis of the Compounds According to the Invention

Material and Methods

All the reagents were obtained from commercial suppliers and used without any further purification. Thin layer chromatography was carried out on TLC silica gel 60 F254 plastic sheets (0.2 mm layer thickness) from Merck. Purification by column chromatography was carried out on silica gel 60 (70-230 mesh ASTM, Merck). The melting points were determined either on a digital device (Electrothermal IA 8103) and are not corrected, or on a Kofler heating bench of type WME (Wagner & Munz). The ¹H, ¹⁹F, and ¹³C nuclear magnetic resonance (NMR) and infrared (IR) spectra confirmed the structures of all of the compounds. The IR spectra were recorded on a Perkin Elmer Spectrum 100 FT-IR spectrometer; and the NMR spectra were recorded, using CDCl₃, CD₃CN, D₂O or DMSO-d₆ as solvent, on a BRUKER AC 300 or 400 spectrometer at 300 or 400 MHz for the ¹H spectra, 75 or 100 MHz spectra for the ¹³C spectra, and 282 or 377 MHz for the ¹⁹F spectra. The chemical shifts (δ) were expressed in parts per million relative to the signal, indirectly (i) with CHCl₃ (δ 7.27) for ¹H; and (ii) with CDCl₃ (δ 77.2) for ¹³C; and directly (iii) with CFCl₃ (internal standard) (δ 0) for ¹⁹F. The chemical shifts are provided in ppm and the peak multiplicities are denoted as follows: s, singlet; br s, broad singlet; d, doublet; dd, doublet of doublets; ddd, doublet of doublets of doublets; t, triplet; q, quartet; quint, quintet; m, multiplet. High-resolution mass spectra (HRMS) were obtained from the “Service central d'analyse de Solaize” (French National Centre for Scientific Research—Solaize) and were recorded on a Waters spectrometer using electrospray ionisation time-of-flight (ESI-TOF) mass spectrometry. Tetramethylsilane (TMS) having the formula Si(CH₃)₄ is used as reference compound for the NMR spectra.

Protocol

Step 1—Synthesis of the Compound having the Formula X-1: The compound having the formula XIV (1.0 equiv.) is dissolved in dichloromethane. The nicotinamide having the formula XV (1.50 equiv.) and the TMSOTf (1.55 equiv.) are added at ambient temperature. The reaction mixture is heated under reflux and stirred until completion of the reaction. The mixture is cooled to ambient temperature and filtered. The filtrate is concentrated to dryness so as to give crude NR (nicotinamide riboside) tetraacetate having the formula X-1.

Step 2—Synthesis of the Compound Having the Formula X: The crude NR tetraacetate having the formula X-1 is dissolved in methanol and cooled to −10° C. This is followed by addition of 4.6 M ammonia in methanol (3.0 equivalents) at −10° C. and the mixture is stirred at this temperature until completion of the reaction. Dowex HCR (H⁺) is added until a pH of 6-7 is attained. The reaction mixture is heated to 0° C. and filtered. The resin is washed with a mixture of methanol and acetonitrile. The filtrate is concentrated until it becomes dry. The residue is dissolved in acetonitrile and concentrated to solid content dryness. The residue is dissolved in acetonitrile so as to give a solution of crude nicotinamide riboside triflate having the formula X.

Step 3—Synthesis of the Compound having the Formula XI: The solution of crude NR nicotinamide riboside triflate in acetonitrile is diluted with trimethyl phosphate (10.0 equivalents). The acetonitrile is distilled under vacuum and the mixture is cooled to −10° C. Phosphorus oxychloride (4.0 equiv.) is added at −10° C. and the mixture is stirred at −10° C. until completion of the reaction.

Step 4 and Step 5: Synthesis of the Compound having the Formula Ia-A: The mixture is hydrolysed by adding a 50/50 mixture of acetonitrile and water, followed by the addition of methyl tert-butyl ether (or tert-butyl methyl ether). The mixture is filtered and the solid is dissolved in water. The aqueous solution is neutralised by adding sodium bicarbonate and extracted with dichloromethane. The aqueous layer is concentrated to dryness so as to give a crude mixture of NMN (Compound I-A) and the compound having the formula Ia-A.

Isolation of the Compound having the Formula Ia-A (β,β diNMN): The NMN and the compound having the formula Ia-A are separated by purification on Dowex 50 w×8 with elution of water. The fractions containing the compound having the formula Ia-A are concentrated to solid content dryness. The residue is purified by column chromatography on silica gel (isopropanol/water gradient). The pure fractions are combined and concentrated. The residue is lyophilised so as to give the Compound I-a-A in the form of a beige solid.

³¹P NMR: δ (ppm, reference 85% H₃PO₄: 0 ppm in D₂O)=−11.72; ¹H NMR: δ (ppm, reference TMS: 0 ppm in D₂O)=4.20 (ddd, J_{H-H}=11.9, 3.5, 2.4 Hz, 2H), 4.35 (ddd, J_{H-H}=11.9, 3.9, 2.2 Hz, 2H), 4.43 (dd, J_{H-H}=5.0, 2.6 Hz, 2H), 4.53 (t, J_{H-H}=5.0 Hz, 2H), 4.59 (m, 2H), 6.16 (d, J_{H-H}=5.4 Hz, 2H), 8.26 (dd, J_{H-H}=8.1, 6.3 Hz, 2H), 8.93 (d, J_{H-H}=8.1 Hz, 2H), 9.25 (d, J_{H-H}=6.2 Hz, 2H), 9.41 (s, 2H); ¹³C NMR: δ (ppm, reference TMS: 0 ppm in D₂O)=64.84 (CH₂), 70.73 (CH), 77.52 (CH), 87.11 (CH), 99.88 (CH), 128.65 (CH), 133.89 (Cq), 139.84 (CH), 142.54 (CH), 146.04 (CH), 165.64 (Cq); MS (ES+): m/z=122.8 [Mnicotinamide+H]+, 650.8 [M+H]+.

Synthesis of the Compound Having the Formula Ia-B (α,β Di-NMN)

Phosphorus oxychloride (3.0 eq.) is added to trimethylphosphate (20.0 eq.) at −5° C. β-NR chloride (1.0 eq.) is added in portions at −5° C. and the reaction mixture is stirred overnight at −5° C. Morpholine (3.0 eq.) is added dropwise at −10/0° C. and the mixture is stirred for 2-3 hrs. α-NMN (1.0 eq.) is then added in portions at −5° C. and the reaction mixture is stirred at −5° C. overnight. The hydrolysis is carried out by dropwise addition of water (5 vol.) at −10/0° C. and the mixture is stirred until complete homogenisation at 10-15° C. The reaction mixture is then extracted with dichloromethane (6*10 vol.) and the aqueous phase is neutralised by elution through the formate resin Purolite A600E (theoretical quantity to neutralise the HCl originating from POCl₃). The eluate is then concentrated in vacuo at 45/50° C. in order to give the crude containing the compound having the formula Ia-B. The water elution with the H⁺ resin Dowex 50 w×8 100-200 mesh makes it possible to remove certain impurities. The fractions containing the Compound I-B are combined and concentrated in vacuo at 45-50° C. The crude is then purified by preparative chromatography on Luna Polar RP 10 μm stationary phase with elution with an aqueous solution of 10 mM NaH2PO4. The pure fractions are combined and eluted with water on the resin Purolite C100EH H⁺(quantity necessary to completely exchange Na+ by H⁺), then eluted on the resin Purolite A600E acetate (quantity necessary to completely exchange H₂PO₄— by acetate). The eluate is concentrated in vacuo and the residue is lyophilised in order to give the Compound Ia-B in the form of a white solid.

³¹P NMR: δ (ppm, reference 85% H₃PO₄: 0 ppm in D₂O)=−11.87, −11.69, −11.46, −11.29; ¹H NMR: 6 (ppm, reference TMS: 0 ppm in D₂O)=4.10 (ddd, J=11.1, 6.1, 3.1 Hz, 1H), 4.15-4.25 (m, 2H), 4.36 (ddd, J=12.2, 4.4, 2.4 Hz, 1H), 4.40 (dd, J=4.9, 2.4 Hz, 1H), 4.44 (dd, J=5.0, 2.7 Hz, 1H), 4.53 (t, J=5.0 Hz, 1H), 4.5 (m, 1H), 4.85 (m, 1H), 4.92 (t, J=5.3 Hz, 1H), 6.15 (d, J=5.5 Hz, 1H), 6.51 (d, J=5.7 Hz, 1H), 8.14 (dd, J=8.0, 6.3 Hz, 1H), 8.26 (dd, J=8.1, 6.3 Hz, 1H), 8.88 (d, J=8.1 Hz, 1H), 8.92 (d, J=8.1 Hz, 1H), 9.02 (d, J=6.3 Hz, 1H), 9.24 (s, 1H), 9.26 (d, J=6.4 Hz, 1H), 9.40 (s, 1H); ¹³C NMR: δ (ppm, reference TMS: 0 ppm in D₂O)=64.83, 64.87 (CH₂), 65.30, 65.35 (CH₂), 70.65 (CH), 70.74 (CH), 71.92 (CH), 77.51 (CH), 87.03, 87.10 (CH), 87.19, 87.26 (CH), 96.57 (CH), 99.83 (CH), 126.89 (CH), 128.54 (CH), 132.44 (Cq), 133.81 (Cq), 139.85 (CH), 140.92 (CH), 142.50 (CH), 143.49 (CH), 145.06 (CH), 145.97 (CH), 165.64 (Cq), 165.88 (Cq); MS (ES+): m/z=122.8 [Mnicotinamide+H]+, 650.9 [M+H]+.

Synthesis of the Compound Having the Formula Ia-C(α,α Di-NMN)

Phosphorus oxychloride (3.0 eq.) is added to trimethylphosphate (20.0 eq.) at −5° C. α-NR chloride (1.0 eq.) is added portionwise at −5° C. and the reaction mixture is stirred overnight at −5° C. Morpholine (3.0 eq.) is added dropwise at −10/0° C. and the mixture is stirred for 2-3 hrs. α-NMN (1.0 eq.) is then added in portions at −5° C. and the reaction mixture is stirred at −5° C. overnight. The hydrolysis is carried out by dropwise addition of water (5 vol.) at −10/0° C. and the mixture is stirred until complete homogenisation at 10-15° C. The reaction mixture is then extracted with dichloromethane (6*10 vol.) and the aqueous phase is neutralised by elution through the formate resin Purolite A600E (theoretical quantity to neutralise the HCl originating from POCl₃). The eluate is then concentrated in vacuo at 45/50° C. to give the crude containing the compound having the formula Ia-C. The water elution with H⁺ resin Dowex 50 w×8 100-200 mesh makes it possible to remove certain impurities. The fractions containing the Compound I-C are combined and concentrated in vacuo at 45-50° C. The crude is then purified by preparative chromatography on Luna Polar RP 10 μm stationary phase with elution with an aqueous solution of 10 mM NaH₂PO₄. The pure fractions are combined and eluted with water on resin Purolite C100EH H⁺(quantity necessary to completely exchange Na+ by H⁺), then eluted on on the resin Purolite A600E acetate (quantity necessary to completely exchange H₂PO₄— by acetate). The eluate is concentrated in vacuo and the residue is freeze-dried in order to give the Compound Ia-C in the form of a white solid.

³¹P NMR: δ (ppm, reference 85% H₃PO₄: 0 ppm in D₂O)=−11.40; ¹H NMR: δ (ppm, reference TMS: 0 ppm in D₂O)=4.14 (ddd, J=11.4, 3.4, 2.8 Hz, 2H), 4.23 (ddd, J=11.6, 3.3, 2.8 Hz, 2H), 4.44 (dd, J=4.8, 2.3 Hz, 2H), 4.88 (m, 2H), 4.96 (t, J=5.3 Hz, 2H), 6.54 (d, J=5.7 Hz, 2H), 8.15 (dd, J=8.1, 6.2 Hz, 2H), 8.89 (d, J=8.1 Hz, 2H), 9.05 (d, J=6.3 Hz, 2H), 9.26 (s, 2H); ¹³C NMR: δ (ppm, reference TMS: 0 ppm in D₂O)=65.37 (CH₂), 70.70 (CH), 71.95 (CH), 87.30 (CH), 96.62 (CH), 126.91 (CH), 132.45 (Cq), 140.94 (CH), 143.52 (CH), 145.07 (CH), 165.90 (Cq); MS (ES+): m/z=122.7 [Mnicotinamide+H]+, 650.8 [M+H]+.

### Example 2: Study of the Efficacy of Compound I-A (NMN Beta)

A satisfaction study was carried out on a group of 12 volunteers, aged 42±8 years, consisting of seven female and five male subjects. The main objective of this study was to evaluate the level of satisfaction of subjects with regard to the evolution of their lumbalgia (low back pain) during the morning and/or evening application of a composition according to the invention containing 5% by weight of NMN.

The average BMI of the participants was 25.3 t 4.4, half of them were overweight (50%), 41.7% were normal weight, and the rest of the subjects were obese (8.3%). More specifically, five participants were of normal weight, six participants were overweight, and one participant was obese. None of these patients presented with a chronic pathology such as an inflammatory pathology altering their cartilage, muscles, tendons, ligaments or bones, or requiring surgery.

The duration of the existence of pains in the lumbar spine region was on average 4±3 years (ie 45 months) while the current pains of the subjects dated back to 2±3 years pre-baseline (before inclusion). These pains occurred mostly spontaneously (58.3%). More precisely, seven participants presented with a lumbalgia of spontaneous origin, one participant presented with a lumbalgia resulting from a physical or sporting activity, two participants attributed their lumbalgia to the practice of gardening, and two other participants attributed their lumbalgia to another of these causes. The participants therefore all had chronic lumbalgia.

A composition in the form of an oil-in-water emulsion comprising 5% NMN was formulated as follows, the ingredients being designated by their INCI (International Nomenclature of Cosmetic Ingredients) name: Aqua, Paraffinum liquidum, Cetyl alcohol, Glyceryl stearate, Benzyl PCA, Ceteareth-20, Ceteareth-12, Cetyl Palmitate, Cocoglycerides, Cetearyl alcohol, Sodium Hydroxide, NMN. The composition was prepared according to any method well known to the person skilled in the art.

The mass percentages are calculated by relating the mass of the ingredient to the total mass of the composition, then by multiplying by 100.

The study took place over 10 days. At baseline, ie upon inclusion (DO), the selected subjects provide their demographic characteristics (age, weight, height), indicate the period of persistence and intensity of the pain on a Visual Analogue Scale, and complete the WOMAC and Lequesne questionnaires. These different scales were used in order to assess by different means the effect of NMN on lumbalgia.

The lumbalgia at baseline was assessed at an average of 73.4±7.6 on a Visual Analogue Scale (VAS) ranging from 0 (no pain) to 100 (intolerable pain).

At baseline, the “stiffness” assessment criterion of the WOMAC questionnaire was the most significant and amounted to 64.2±21.0, the “pain” assessment criterion amounted to 59.9±13.1 and the “function” assessment criterion amounted to 57.1±15.5. The total WOMAC score was 58.3±14.8 at baseline. The higher the WOMAC score, the greater the functional pain interference.

The Lequesne algo-functional index is used for the clinical follow-up of lumbalgia in the present study. The Lequesne score on inclusion was on average 8.3±2.5, and 3 of the subjects had a score greater than or equal to 10 (16.7%), which demonstrates a very significant or indeed even intolerable impairment.

Over the subsequent 9 day period, each evening the subjects fill in the Visual Analogue Scale of pain, ensuring any discomfort or problems arising or the taking of analgesics are reported therein.

On day 10, the volunteers complete the WOMAC questionnaire, the Lequesne questionnaire, the Visual Analogue Scale (VAS) of pain, indicating the perceived improvement in lumbalgia pain relief as measured by the PGI-I index (abbreviation for “Patient Global Improvement Impression”), satisfaction with regard to evolution of the lumbalgia on a Likert scale as well as the ease of application and penetration of the composition, assessment of the texture and odour of the composition, likelihood of re-use thereof in the event of recurrence of a similar pain, and recommendation of use to third parties presenting with pain of similar nature. The PGI-I index is an index that serves as means for evaluating the response to a treatment. The Likert scale is a psychometric tool used for measuring an attitude in individuals, which consists of one or more statements for which the individual responding expresses their degree of agreement or disagreement.

During the study, product compliance was optimal with a compliance rate of 97.7%. Indeed, during the 9 days of follow-up, almost all of the subjects applied the composition twice a day, as shown in Table 1.

TABLE 3

| | Time of application | | | | | | | | |
|---|---|---|---|---|---|---|---|---|---|
| | D | D | D | D | D | D | D | D | D |
| | 1 | 2 | 3 | 4 | 5 | 6 | 7 | 8 | 9 |
| Previous | 10 | 12 | 11 | 12 | 12 | 12 | 11 | 12 | 12 |
| evening and | | | | | | | | | |
| morning | | | | | | | | | |
| Previous | 0 | 0 | 1 | 0 | 0 | 0 | 1 | 0 | 0 |
| evening | | | | | | | | | |
| Morning | 2 | 0 | 0 | 0 | 0 | 0 | 0 | 0 | 0 |
| Total | 12 | 12 | 12 | 12 | 12 | 12 | 12 | 12 | 12 |

The lumbalgia pain, as measured by the VAS, decreased steadily over the 10 days of application of the product, dropping from 73.4±7.6 at baseline to 30.8±22.6, that is to say a significant reduction of 58.7±29.2% (p<0.0001, calculated with a Student's t test). The mean time period in order to obtain an initial 50% reduction in pain relative to baseline was 5.0±2.9 days. The results expressed as mean and standard deviation, on day-by-day basis, and for all of the volunteers, are summarised in Table 2 below:

TABLE 4

| | | Standard | | Mini- | Maxi- | | Signif- |
|---|---|---|---|---|---|---|---|
| Days | Mean | deviation | Median | mum | mum | p-value | icance |
| D 0 | 73.4 | 7.6 | 72.5 | 64.0 | 84.0 | NA | NA |
| D 1 | 61.1 | 14.8 | 60.5 | 34.0 | 81.0 | 0.0163 | <0.05 |
| D 2 | 56.8 | 20.3 | 59.0 | 22.0 | 83.0 | 0.0138 | <0.05 |
| D 3 | 52.3 | 22.1 | 57.5 | 14.0 | 81.0 | 0.0031 | <0.01 |
| D 4 | 49.4 | 18.9 | 51.0 | 19.0 | 79.0 | 0.001 | <0.001 |
| D 5 | 45.7 | 19.1 | 45.5 | 17.0 | 76.0 | 0.0001 | <0.001 |
| D 6 | 42.4 | 18.5 | 42.5 | 17.0 | 71.0 | <0.0001 | <0.0001 |
| D 7 | 45.2 | 25.2 | 48.0 | 4.0 | 93.0 | 0.0017 | <0.01 |
| D 8 | 35.8 | 24.8 | 38.0 | 4.0 | 96.0 | 0.0002 | <0.001 |
| D 9 | 28.3 | 20.2 | 26.0 | 0.0 | 77.0 | <0.0001 | <0.0001 |
| D 10 | 30.8 | 22.6 | 30.0 | 5.0 | 77.0 | <0.0001 | <0.0001 |

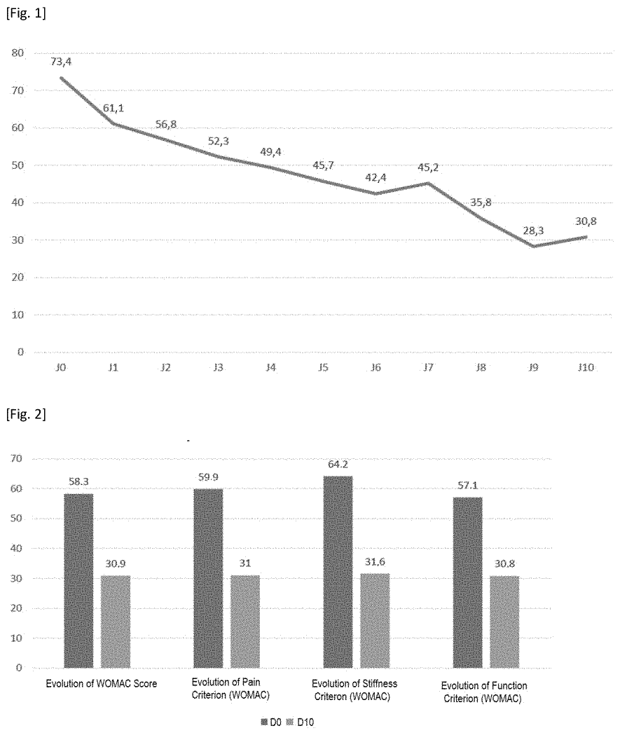

These results are in addition represented by the graph in FIG. 1. As may be seen in FIG. 1 and in view of the results in Table 2 the pain felt by the patients decreased on average by 58.7%.

After 10 days of application of the composition according to the invention, the “pain” criterion of the WOMAC fell from 59.9±13.1 at baseline to 31.0±21.2 at the end of the study, which is a significant reduction of 50.1±31.3% (p<0.001) as may be seen in FIG. 2. The reductions for the other assessment criteria were also significant, declining from 64.2±21.0 to 31.6±25.0 for the “stiffness” criterion of the WOMAC (reduction of 52.5±32.9%, p<0.001), and from 57.1±15.5% to 30.8±21.8 for the “function” criterion of the WOMAC (reduction of 47.5±35.6%, p<0.01). The total WOMAC score also decreased significantly from 58.3±14.8 to 30.9±21.7, which is a decrease of 48.6±33.7% (p<0.001).

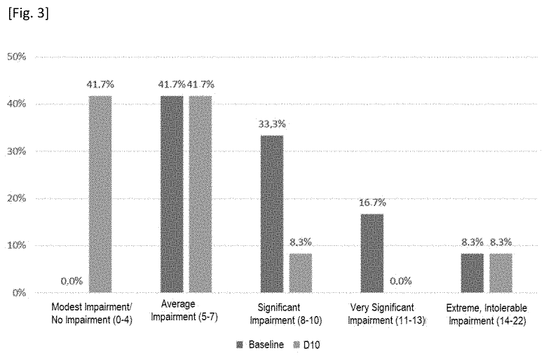

As may be seen in FIG. 3, the Lequesne algo-functional score decreased significantly from baseline to study endpoint, decreasing from 8.3±2.5 to 5.3±3.8 (p<0.001) which is a 38.8% reduction in the score. At the end of the study, more than three quarters of the subjects (83.4%) no longer had any impairment or had only a modest or slight impairment.

At the end of the study, 91.7% of the subjects experienced improvement, characterised as: ‘considerable’—3 subjects (25.0%); ‘a lot’—4 subjects (33.3%); and ‘slight’—4 subjects (33.3%). Only one subject showed no improvement. Almost all (91.7%) of the participants were satisfied with the evolution of their chronic lumbalgia, including 33.3% who were very satisfied.

From an organoleptic standpoint, all the patient responses confirmed that the composition: was easy to apply (66.7%—very easy); penetrated easily into the skin (58.3%—very easily); had a pleasant texture (33.3%—very pleasant); and a pleasant smell (58.3%—pleasant and 8.3%—very pleasant).

All of the patients confirmed their likelihood of: reusing the composition in the event of recurrence of a similar lumbalgia (75.0%—very certainly); and recommending it to others experiencing similar pain in the lumbar region (50.0%—very certainly).

One patient took paracetamol on D5 because of lumbar pain.

The volunteer participants experienced neither any adverse side effects following the use of the composition according to the invention nor developed any allergies.

NMN, a pharmaceutically acceptable derivative thereof, or a pharmaceutically acceptable salt thereof, as well as compositions that comprise the same are therefore effective in reducing back pain, and in particular chronic lumbalgia. Furthermore, the use of NMN and of the composition comprising NMN in accordance with the invention enabled participants to avoid having to resort to using their usual treatment to relieve their lumbar pain, or at the very least to reduce the need to use conventional therapies. Although the demonstration was carried out for the treatment of lumbalgia, the results may be transposed to the treatment of back pain. The present invention therefore provides a safe and effective alternative to conventional therapies for back pain, and in particular for lumbalgia.

1. Nicotinamide mononucleotide (NMN), a pharmaceutically acceptable derivative thereof, or a pharmaceutically acceptable salt thereof, for use thereof via topical administration in the prevention and/or treatment of a back pain.

2. Nicotinamide mononucleotide (NMN), a pharmaceutically acceptable derivative thereof, or a pharmaceutically acceptable salt thereof, for use thereof according to claim 1 in which the pharmaceutically acceptable derivative of NMN is dihydronicotinamide mononucleotide (NMN-H), alpha-NMN, a compound having the formula (I): 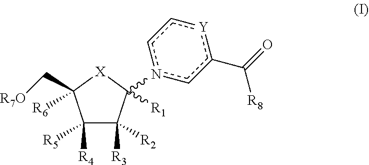
or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, and P(S)R₉R₁₀ and 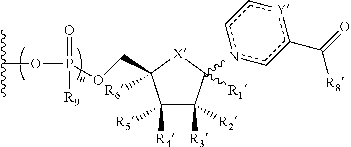
where n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂SO₂; wherein each of Ria is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃; [symbol]
represents a single or a double bond along Y; and [symbol]
represents the alpha or beta anomer depending on the position of R₁;
or
a compound having the formula (Ia): 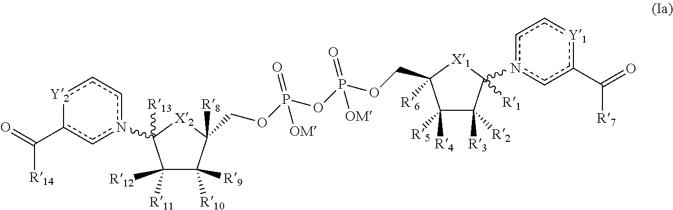
or one of the: stereoisomers, salts, hydrates, solvates, or crystals thereof, in which:
X′₁ and X′₂ are independently selected from among O, CH₂, S, Se, CHF, CF₂, and C═CH₂;
R′₁ and R′₁₃ are independently selected from among H, azido, cyano, a C₁-C₈ alkyl, a C₁-C₈ thio-alkyl, a C₁-C₈ heteroalkyl, and OR, wherein R is selected from H and a C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH2 group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′1 and Y′2 are independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
and combinations thereof, for use thereof via topical administration in the prevention and/or treatment of a back pain.

3. Nicotinamide mononucleotide (NMN), a pharmaceutically acceptable derivative thereof, or a pharmaceutically acceptable salt thereof, for use thereof according to claim 2 in which the pharmaceutically acceptable derivative of NMN is selected from among: Compound I-B, Compound I-C, Compound I-D, Compound I-E, Compound I-F, Compound I-G, Compound I-H, Compound I-I, Compound I-J, preferably Compound I-B, Compound I-C, Compound I-D, Compound I-F, and combinations thereof from Table 1.

4. Nicotinamide mononucleotide (NMN), a pharmaceutically acceptable derivative thereof, or a pharmaceutically acceptable salt thereof, for use thereof according to claim 2 is selected from among the compounds Ia-A to Ia-I, preferably from among the compound having the formula Ia-B, the compound having the formula Ia-C, the compound having the formula Ia-E, the compound having the formula Ia-F, the compound having the formula Ia-H, the compound having the formula Ia-I and the compound having the formula Ia-G of Table 2.

5. Nicotinamide mononucleotide (NMN), a pharmaceutically acceptable derivative thereof, or a pharmaceutically acceptable salt thereof, for use thereof according to claim 1, in which the back pain is a cervicalgia, a dorsalgia, or a lumbalgia; and preferably a chronic lumbalgia.

6. Nicotinamide mononucleotide (NMN), a pharmaceutically acceptable derivative thereof, or a pharmaceutically acceptable salt thereof, for use thereof according to claim 1 in which the back pain is due to one of the pathologies selected from among: injury to a muscle, injury to a ligament, injury to a tendon, degeneration of the intervertebral discs in the vertebrae, a herniated disc in the vertebrae, a pain that is gynecological in origin, spondylolisthesis, arthritis, osteoarthritis, osteoporosis of the vertebral column, osteoporosis-related fracture, an abdominal aortic aneurysm, a tumour, an infection, an inflammation, facet joint injuries, intervertebral disc injuries, regional or global spinal stability-related disorders, spinal deformity, muscular contraction in the vertebrae, or combinations thereof.

7. Nicotinamide mononucleotide (NMN), a pharmaceutically acceptable derivative thereof, or a pharmaceutically acceptable salt thereof, for use thereof in combination with at least one other therapeutic agent.

8. Nicotinamide mononucleotide (NMN), a pharmaceutically acceptable derivative thereof, or a pharmaceutically acceptable salt thereof, for use thereof wherein the at least one therapeutic agent may be an analgesic, a non-steroidal anti-inflammatory drug, cortisone, a cortisone derivative, a muscle relaxant, or combinations thereof.

9. A composition comprising nicotinamide mononucleotide (NMN), a pharmaceutically acceptable derivative thereof, or a pharmaceutically acceptable salt thereof, and at least one pharmaceutically acceptable excipient, for use thereof via topical administration, in the prevention and/or treatment of a back pain.

10. The composition according to claim 9, in which the pharmaceutically acceptable derivative is selected from among dihydronicotinamide mononucleotide (NMN-H), alpha-NMN, a compound having the formula (I): 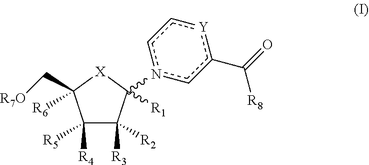
or one of the pharmaceutically acceptable: stereoisomers, salts, hydrates, solvates, or crystals thereof, in which:
X is selected from among O, CH₂, S, Se, CHF, CF₂ and C═CH₂;
R₁ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₂, R₃, R₄ and R₅ are selected independently of one another, from among H, halogen, azido, cyano, hydroxyl, (C₁-C₁₂) alkyl, (C₁-C₁₂) thio-alkyl, (C₁-C₁₂) heteroalkyl, (C₁-C₁₂) haloalkyl, and OR; wherein R is selected from among H, (C₁-C₁₂) alkyl, C(O)(C₁-C₁₂)alkyl, C(O)NH(C₁-C₁₂)alkyl, C(O)O(C₁-C₁₂)alkyl, C(O)aryl, C(O)(C₁-C₁₂)alkyl aryl, C(O)NH(C₁-C₁₂)alkyl aryl, C(O)O(C₁-C₁₂)alkyl aryl, and C(O)CHR_{AA}NH₂; wherein R_{AA} is a side chain selected from a proteinogenic amino acid;
R₆ is selected from among H, azido, cyano, (C₁-C₈) alkyl, (C₁-C₈) thio-alkyl, (C₁-C₈) heteroalkyl, and OR; wherein R is selected from H and (C₁-C₈) alkyl;
R₇ is selected from among H, P(O)R₉R₁₀, and P(S)R₉R₁₀ and 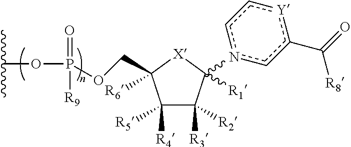
where n is an integer selected from 1 or 3; in which
R₉ and R₁₀ are selected independently of one another, from among OH, OR₁₁, NHR₁₃, NR₁₃R₁₄, a (C₁-C₈) alkyl, a (C₂-C₈) alkenyl, a (C₂-C₈)alkynyl, a (C₃-C₁₀) cycloalkyl, a (C₅-C₁₂) aryl, (C₁-C₈)alkyl aryl, (C₁-C₈) aryl alkyl, (C₁-C₈) heteroalkyl, (C₁-C₈) heterocycloalkyl, a heteroaryl, and NHCHR_{A}R_{A′}C(O)R₁₂; in which:
R₁₁ is selected from among a group: (C₁-C₁₀) alkyl, (C₃-C₁₀) cycloalkyl, (C₅-C₁₈) aryl, (C₁-C₁₀) alkylaryl, substituted (C₅-C₁₂) aryl, (C₁-C₁₀) heteroalkyl, (C₃-C₁₀) heterocycloalkyl, (C₁-C₁₀) haloalkyl, a heteroaryl, —(CH₂)ₙC(O)(C₁-C₁₅)alkyl, —(CH₂)ₙOC(O)(C₁-C₁₅)alkyl, —(CH₂)ₙOC(O)O(C₁-C₁₅)alkyl, —(CH₂)ₙSC(O)(C₁-C₁₅)alkyl, —(CH₂)ₙC(O)O(C₁-C₁₅)alkyl, and —(CH₂)ₙC(O)O(C₁-C₁₅)alkyl aryl; wherein n is an integer selected from 1 to 8; P(O)(OH)OP(O)(OH)₂; halogen, nitro, cyano, C₁-C₆ alkoxy, C₁-C₆ haloalkoxy, —N(R₁₁ₐ)₂, C₁-C₆ acylamino, —COR_{11b}, —OCOR_{11b}; NHSO₂(C₁-C₆ alkyl), —SO₂N(R₁₁ₐ)₂SO₂; wherein each of R₁₁ₐ is independently selected from H and a (C₁-C₆) alkyl, and R_{11b} is independently selected from OH, C₁-C₆ alkoxy, NH₂, NH(C₁-C₆ alkyl) or N(C₁-C₆ alkyl)₂;
R₁₂ is selected from among H, C₁-C₁₀ alkyl, C₂-C₈ alkenyl, C₂-C₈ alkynyl, C₁-C₁₀ haloalkyl, C₃-C₁₀ cycloalkyl, C₃-C₁₀ heterocycloalkyl, C₅-C₁₈ aryl, C₁-C₄ alkylaryl, and C₅-C₁₂ heteroaryl; wherein the said aryl or heteroaryl groups are optionally substituted with one or two groups selected from among halogen, trifluoromethyl, C₁-C₆ alkyl, C₁-C₆ alkoxy, and cyano; and
R_{A} and R_{A′} are independently selected from among H, a (C₁-C₁₀) alkyl, (C₂-C₁₀) alkenyl, (C₂-C₁₀) alkynyl, (C₃-C₁₀) cycloalkyl, (C₁-C₁₀) thio-alkyl, (C₁-C₁₀) hydroxylalkyl, (C₁-C₁₀) alkylaryl, and (C₅-C₁₂) aryl, (C₃-C₁₀) heterocycloalkyl, a heteroaryl, —(CH₂)₃NHC(═NH)NH₂, (1H-indol-3-yl)methyl, (1H-imidazol-4-yl)methyl, and a side chain selected from among a proteinogenic amino acid or a non-proteinogenic amino acid; wherein the said aryl groups are optionally substituted with a group selected from among hydroxyl, (C₁-C₁₀) alkyl, (C₁-C₆) alkoxy, a halogen, a nitro, and a cyano; or
R₉ and R₁₀ form, together with the phosphorus atoms to which they are attached, a 6-membered ring in which —R₉—R₁₀— represents —CH₂—CH₂—CHR—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano; or
R₉ and R₁₀ form, together with the phosphorus atoms to which they are attached, a 6-membered ring in which —R₉—R₁₀— represents —O—CH₂—CH₂—CHR—O—; wherein R is selected from among H, a (C₅-C₆) aryl group, and (C₅-C₆) heteroaryl group, wherein the said aryl or heteroaryl groups are optionally substituted by a halogen, trifluoromethyl, a (C₁-C₆) alkyl, a (C₁-C₆) alkoxy, and cyano;
R₈ is selected from among H, OR, NHR₁₃, NR₁₃R₁₄, NH—NHR₁₃, SH, CN, N₃, and halogen; wherein R₁₃ and R₁₄ are selected independently of one another, from among H, (C₁-C₈) alkyl, (C₁-C₈) alkyl aryl, and —CR_{B}R_{C}—C(O)—OR_{D} in which R_{B} and R_{C} are independently a hydrogen atom, a (C₁-C₆) alkyl, a (C₁-C₆) alkoxy, benzyl, indolyl, or imidazolyl; where the (C₁-C₆) alkyl and the (C₁-C₆) alkoxy may be optionally and independently of one another substituted by one or more of the halogen, amino, amido, guanidyl, hydroxyl, thiol, or carboxyl groups, and the benzyl group is optionally substituted by one or more halogen or hydroxyl groups; or R_{B} and R_{C} form, together with the carbon atom to which they are attached, a C₃-C₆ cycloalkyl group optionally substituted by one or more halogens, amino, amido, guanidyl, hydroxyl, thiol, and carboxyl; and R_{D} is a hydrogen, a (C₁-C₆) alkyl, a (C₂-C₆) alkenyl, a (C₂-C₆) alkynyl, or a (C₃-C₆) cycloalkyl;
Y is selected from among CH, CH₂, C(CH₃)₂ and CCH₃;
n is an integer selected from 1 to 3; [symbol]
represents a single or a double bond along Y; and [symbol]
represents the alpha or beta anomer depending on the position of R₁;
or
a compound having the formula (Ia): 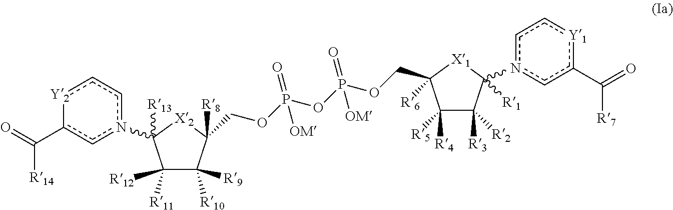
or one of the: stereoisomers, salts, hydrates, solvates, or crystals thereof, in which:
X′₁ and X′₂ are independently selected from among O, CH₂, S, Se, CHF, CF₂, and C═CH₂;
R′₁ and R′₁₃ are independently selected from among H, azido, cyano, a C₁-C₈ alkyl, a C₁-C₈ thio-alkyl, a C₁-C₈ heteroalkyl, and OR, wherein R is selected from H and a C₁-C₈ alkyl;
R′₂, R′₃, R′₄, R′₅, R′₉, R′₁₀, R′₁₁, R′₁₂ are independently selected from among H, a halogen, an azido, a cyano, a hydroxyl, a C₁-C₁₂ alkyl, a C₁-C₁₂ thioalkyl, a C₁-C₁₂ hetero-alkyl, a C₁-C₁₂ haloalkyl, and OR; wherein R may be selected from among H, a C₁-C₁₂ alkyl, a C(O)(C₁-C₁₂) alkyl, a C(O)NH(C₁-C₁₂) alkyl, a C(O)O(C₁-C₁₂) alkyl, a C(O) aryl, a C(O)(C₁-C₁₂) aryl, a C(O)NH(C₁-C₁₂) alkyl aryl, a C(O)O(C₁-C₁₂) alkyl aryl, or a C(O)CHR_{AA}NH₂ group; wherein R_{AA} is a side chain selected from a proteinogenic amino acid;
R′₆ and R′₈ are independently selected from among H, an azido, a cyano, a C₁-C₈ alkyl and OR, wherein R is selected from H and a C₁-C₈ alkyl;
R′₇ and R′₁₄ are independently selected from among H, OR, NHR, NRR′, NH—NHR, SH, CN, N₃ and a halogen; wherein R and R′ are independently selected from H and a (C₁-C₈) alkyl aryl;
Y′1 and Y′2 are independently selected from among CH, CH₂, C(CH₃)₂, or CCH₃;
M′ is selected from H or a suitable counter ion; [symbol]
represents a single or double bond depending on Y′₁ and Y′₂; and [symbol]
represents an alpha or beta anomer depending on the position of R′₁ and R′₁₃;
and combinations thereof, for use thereof via topical administration in the prevention and/or treatment of a back pain.

11. The composition according to claim 9 that comprises nicotinamide mononucleotide (NMN), a pharmaceutically acceptable derivative thereof, or a pharmaceutically acceptable salt thereof, in an amount comprised between 0.05% and 15% by weight, preferably between 1 and 10% by weight, on a more preferred basis between 3 and 5% by weight relative to the total weight of the composition.

12. The composition according to claim 9, which is in the form of a gel, a solution, a water-in-oil emulsion, an oil-in-water emulsion, an oil, a cream, an ointment, or a liniment; on a more preferred basis in the form of an oil-in-water emulsion.

13. The composition according to claim 9 further comprising at least one additional therapeutic agent.

14. The composition according to claim 10, in which the pharmaceutically acceptable derivative of NMN is selected from among: Compound I-B, Compound I-C, Compound I-D, Compound I-E, Compound I-F, Compound I-G, Compound I-H, Compound I-I, Compound I-J; preferably Compound I-B, Compound I-C, Compound I-D, Compound I-F from Table 1; Compound Ia-A, the compound having the formula Ia-B, the compound having the formula Ia-C, the compound having the formula Ia-E, the compound having the formula Ia-F, the compound having the formula Ia-H, the compound having the formula Ia-I, the compound having the formula Ia-G from Table 2, and combinations thereof.

15. The composition according to claim 13, in which the at least one therapeutic agent may be an analgesic, a non-steroidal anti-inflammatory drug, cortisone, a cortisone derivative, a muscle relaxant, or combinations thereof.
